(19) United States
(12) Patent Application Publication
(10) Pub. No.: US 2025/0339106 A1
(43) Pub. Date: Nov. 6, 2025

(54) SYSTEMS AND METHODS FOR ENSURING AN ACCURACY OF AN ANALYTE DEVICE BY PERFORMING ALERT STATE BACKFILLING

(71) Applicant: Dexcom, Inc., San Diego, CA (US)

(72) Inventors: Rasoul YOUSEFI, San Diego, CA (US); Douglas S. KANTER, San Diego, CA (US); Stefan M. ROBERT, San Diego, CA (US)

(21) Appl. No.: 19/198,218

(22) Filed: May 5, 2025

Related U.S. Application Data
(60) Provisional application No. 63/643,251, filed on May 6, 2024.

(51) Int. Cl. A61B 5/00; A61B 5/145

(52) U.S. Cl. CPC A61B 5/742; A61B 5/14546

(57) ABSTRACT

The present disclosure provides systems, methods, and devices for recovery and/or adjustment of an alert state of a continuous analyte monitoring system following signal loss events associated with wireless connections between an analyte sensor system and a display device. Certain embodiments of the present disclosure describe a continuous analyte monitoring system that may retrospectively analyze cached backfill data and update a current alert state and associated conditions and/or settings on a display device after a signal loss event according to one or modes of operation.

### CROSS-REFERENCE TO RELATED APPLICATIONS

This application claims priority to and benefit of U.S. Provisional Application No. 63/643,251, filed May 6, 2024, which is hereby assigned to the assignee hereof and hereby expressly incorporated by reference in their entirety as if fully set forth below and for all applicable purposes.

### BACKGROUND

Diabetes mellitus is a metabolic condition relating to the production or use of insulin by the body. Insulin is a hormone that allows the body to use glucose for energy, or store glucose as fat.

When a person eats a meal that contains carbohydrates, the digestive system absorbs nutrients, ultimately depositing glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), skin ulcers, and kidney damage. Hyperglycemia can also lead to acute problems, such as diabetic ketoacidosis—a state in which the body becomes excessively acidic due to the production of excess ketones, or body acids. The state of having lower than normal blood glucose levels is called “hypoglycemia.” Severe hypoglycemia can lead to damage of the heart muscle, neurocognitive dysfunction, and in certain cases, acute crises that can result in seizures or even death.

A patient living with diabetes can receive insulin to manage blood glucose levels. Insulin can be received, for example, through a manual injection with a needle. Wearable insulin pumps are also available. Diet and exercise also affect blood glucose levels.

Diabetes conditions are sometimes referred to as “Type 1” and “Type 2”. A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

Patients with diabetes can benefit from real-time diabetes management guidance, as determined based on a physiological state of the patient, in order to stay within a target glucose range and avoid physical complications. In certain cases, the physiological state of the patient is determined using monitoring systems that measure glucose levels, which inform the identification and/or prediction of adverse glycemic events, such as hyperglycemia and hypoglycemia, and the type of guidance provided to the patient.

For example, such monitoring systems may utilize a continuous glucose monitor (CGM) to measure a patient's glucose levels over time. The measured glucose levels may then be processed by the monitoring system to identify and/or predict adverse glycemic events, and/or to provide guidance to the patient for treatment and or actions to abate or prevent the occurrence of such adverse glycemic events. For example, trends, statistics, or other metrics may be derived from the glucose levels and used to identify and/or predict adverse glycemic events. Or, in certain cases, the glucose levels themselves may be used to identify and/or predict adverse glycemic events.

Generally, a continuous glucose monitor wirelessly transmits raw or minimally processed glucose data for subsequent processing, analysis, and/or display at one or more remote devices, which can include a display device, a server, or any other types of communication devices. Periodically, the wireless connection between the continuous glucose monitor and the one or more remote devices may be disrupted for a variety of reasons, which leads to a loss of signal at the one or more remote devices, and thus, a gap in glucose level measurements received by the one or more remote devices from the continuous glucose monitor. These gaps in glucose level measurements can negatively impact the performance of a monitoring system, such as by reducing the accuracy of the guidance provided by diagnostics systems. This reduced guidance accuracy may decrease an amount of trust a patient has in the monitoring system, which may in turn decrease a responsiveness of the patient to guidance provided by the monitoring system, which may negatively impact a health of the patient.

This background is provided to introduce a brief context for the summary and detailed description that follow. This background is not intended to be an aid in determining the scope of the claimed subject matter nor be viewed as limiting the claimed subject matter to implementations that solve any or all of the disadvantages or problems presented above.

### SUMMARY

Aspects of the present disclosure provide systems, methods, and devices for alert state recovery and/or adjustment following signal loss events associated with wireless connections between an analyte sensor system and a display device. In particular, certain embodiments of the present disclosure describe a continuous analyte monitoring system that may retrospectively analyze cached backfill data and update a current alert state and associated conditions and/or settings on a display device after a signal loss event.

Accordingly, certain embodiments herein provide a method for ensuring the accuracy of a display device of an analyte monitoring system, comprising: re-establishing a wireless communication signal between a display device and an analyte sensor system after a signal loss event; upon re-establishing the wireless communication signal, receiving, by the display device, backfill data from the analyte sensor system, the backfill data comprising historical analyte data of the patient collected by the analyte sensor system during the signal loss event; and processing, by the display device, the current analyte data and the backfill data, or the current analyte data and not the backfill data, to determine a current alert state of the display device.

Certain embodiments herein provide an analyte monitoring system, the analyte monitoring system comprising a sensor system, comprising: a continuous analyte sensor configured to measure an analyte concentration of a patient; a sensor electronics module configured to: receive a signal from the continuous analyte sensor that is indicative of the analyte concentration, generate analyte data based on the signal, and transmit, via a wireless transceiver, the analyte data to at least a first display device; and, the first display device in direct wireless communication with the sensor electronics module, comprising: one or more memories, and one or more processors communicatively coupled to the one or more memories, the one or more processors configured to ensure an accuracy of the first display device by determining a current alert state of the first display device based on at least one of received historical analyte data and current analyte data; wherein upon recovery of wireless communication between the first display device and the sensor electronics module after a signal loss event, the historical analyte data generated by the sensor electronics module during the signal loss event is received from the sensor electronics module by the one or more processors; and wherein the currently analyte data generated by the sensor electronics module after the signal loss event is received from the sensor electronics module by the one or more processors.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 illustrates aspects of an example continuous analyte monitoring system used in connection with implementing embodiments of the present disclosure.

FIG. 2 is a diagram conceptually illustrating the continuous analyte monitoring system of FIG. 1, in accordance with certain aspects of the present disclosure.

FIG. 3A is a diagram of a time series of glucose measurements illustrating example conditions for alert states of the continuous analyte monitoring system of FIG. 1, in accordance with certain aspects of the present disclosure.

FIG. 3B is another diagram of a time series of glucose measurements illustrating the triggering of an alert state and an associated timer, in accordance with certain aspects of the present disclosure.

FIG. 4A illustrates an example signal loss scenario with conventional analyte monitoring systems that may result in redundant alerts being generated and output to a patient, in accordance with certain aspects of the present disclosure.

FIG. 4B illustrates an example signal loss scenario with conventional analyte monitoring systems that may result in significant delays in alerts being generated and output to a patient, in accordance with certain aspects of the present disclosure.

FIG. 5 illustrates a flow diagram depicting an example method for recovery of prior alert states and associated conditions and/or settings after a signal loss event, in accordance with certain aspects of the present disclosure.

FIG. 6A illustrates a flow diagram depicting another example method for recovery of prior alert states and associated conditions and/or settings after a signal loss event, in accordance with certain aspects of the present disclosure.

FIG. 6B illustrates a flow diagram depicting a signal loss scenario after which the alert state recovery method of FIG. 6A is implemented, in accordance with certain aspects of the present disclosure.

FIG. 6C illustrates a flow diagram depicting another signal loss scenario after which the alert state recovery method of FIG. 6A is implemented, in accordance with certain aspects of the present disclosure.

FIG. 7 illustrates a flow diagram depicting another example method for recovery of prior alert states and associated conditions and/or settings after a signal loss event, in accordance with certain aspects of the present disclosure.

FIG. 8 illustrates a flow diagram depicting example inputs and outputs of the alert state recovery methods described here.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Aspects of the present disclosure provide systems, methods, and devices for alert state recovery and/or adjustment following signal loss events associated with wireless connections between an analyte sensor system and a display device. For example, an analyte sensor system may be worn by a patient and be configured to continuously measure analyte levels of the patient. These analyte levels may then be wirelessly transmitted from the analyte sensor system to a display device (e.g., smart phone or smart watch) using an antenna system comprising one or more antennas, allowing the patient to conveniently track their analyte levels on an analyte monitoring application running on the display device.

In some embodiments, information packets including the analyte levels of the patient may be transmitted from the analyte sensor system to the display device using a wireless connection established between the analyte sensor system and the display device. Proper reception of the information packets by the display device requires that these information packets be received with a strong signal strength. If a signal strength associated with transmissions on the wireless connection is weak or interrupted, this may lead to the information packets being lost, i.e., not received by the display device.

The wireless connection established between the analyte sensor system and display device may be negatively affected by various factors, such as interference from other devices, a patient setting the display device down and walking a distance from the display device that is beyond a predetermined threshold, the patient placing the analyte sensor system or display device in a particular position or location that blocks the transmission/reception of the information packets, power loss or rebooting of the display device, and the like. Each scenario may lead to a disruption in the wireless connection between the analyte sensor system and the display device, or “signal loss.”

In conventional analyte monitoring systems, upon occurrence of a signal loss event, all prior glucose alerts and their associated patient acknowledgements and timers on the display device are dismissed. In other words, the alert state is reset upon loss of signal between the analyte sensor system and the display device, whether acknowledged or not. Thus, when such analyte monitoring systems recover from the signal loss event, if a prior (e.g., prior to the signal loss event) alert state triggering an alert is still present upon signal recovery, a patient may be forced to re-acknowledge the alert, even if the patient had already acknowledged the alert prior to signal loss. This may occur repetitively if multiple signal loss events occur in succession. Accordingly, the current approach of resetting alert states during signal loss events may lead to alert fatigue and confusion of the user in certain scenarios. This alert fatigue/confusion may decrease the likelihood that a patient responds to future guidance provided by such analyte monitoring systems, which may negatively impact a health of the patient. The generation and presentation of redundant alerts on the display device also presents an unnecessary processing burden (as well as unnecessary memory usage and network bandwidth) on a hardware processor of the display device.

Even further, if a timer for the generation/transmission of an alert is pending prior to a signal loss event, and the alert state triggering the timed alert is still present upon signal recovery, the resetting of the alert state as caused by the signal loss event will also reset the alert timer. In such scenarios, the generation/transmission of the alert may be significantly delayed due to the alert timer being reset, particularly if multiple signal loss events occur in succession and/or a signal loss event occurs towards the end of an alert timer countdown. As a result, a patient may not be aware that they are experiencing an analyte state warranting attention until long after transitioning into the analyte state, which may be dangerous to a health of the patient. Also, if the analyte monitoring system sends one or more signals to automatically administer medicament to a patient in response to a particular alert, the delay of such alert may also delay the sending of such signals (and the subsequent administration of medicament), which may negatively impact the health of the patient.

Accordingly, aspects of the present disclosure provide techniques for avoiding the scenarios described above, as well as other scenarios related to signal loss events between an analyte sensor system and display device. In certain embodiments, these techniques may include the retrospective analysis of backfill data that is cached at the analyte sensor system during a signal loss event and is later transmitted to a display device upon signal recovery. Upon the retrospective analysis of the backfill data, the display device may adjust or update a current alert state and any associated conditions or settings. In certain embodiments, the display device may perform one or more checks (e.g., evaluations) after a signal loss event, the result of such checks determining whether retrospective analysis of the backfill data will be needed to adjust or update the current alert state, and what portions of the backfill data will need to be analyzed.

After the recovery of a signal loss event, the retrospective analysis of backfill data cached at the analyte sensor system avoids unnecessary repetition of alerts and enables the subsequent recovery of prior alert states and associated acknowledgements and timers. This may improve and/or ensure an accuracy of guidance provided by the analyte sensor system, which may in turn increase an amount of trust a patient has in the analyte sensor system. This may increase a responsiveness of the patient to guidance provided by the analyte sensor system, which may positively impact the health of the patient. For example, the patient may be more likely to exercise or administer insulin in response to a hyperglycemic alert, and the patient may be more likely to consume glucose in response to a hypoglycemic event (instead of disregarding such alerts as redundant or inaccurate).

Also, by ensuring that alert timers generated by the analyte sensor system are accurate, a patient may be made aware that they are about to experience an analyte state warranting attention at the correct time (e.g., before transitioning into the analyte state), which may improve a health of the patient. Also, by ensuring proper/accurate alert timer generation by the analyte sensor system, a timeliness of signals sent by the analyte sensor system to automatically administer medicament to a patient in response to such alerts may be ensured, which may improve a health of the patient.

Additionally, performing one or more checks prior to the retrospective analysis of the backfill data (to ensure that such analysis is performed only when necessary) reduces an amount of processing necessary by both a sensor and the display device during the aforementioned alert state recovery. Avoiding the generation and presentation of redundant alerts on the display device also avoids an unnecessary processing burden (as well as unnecessary memory usage and network bandwidth) on a hardware processor of the display device, thereby improving a functioning of such computing hardware. These and other benefits of the present disclosure are described in further detail below.

The details of some example embodiments of the systems, methods, and devices of the present disclosure are set forth in this description and in some cases, in other portions of the disclosure. Other features, objects, and advantages of the disclosure will be apparent to one of skill in the art upon examination of the present disclosure, description, figures, examples, and claims. It is intended that all such additional systems, methods, devices, features, and advantages be included within this description (whether explicitly or by reference), be within the scope of the present disclosure, and be protected by one or more of the accompanying claims.

### System Overview and Example Configurations

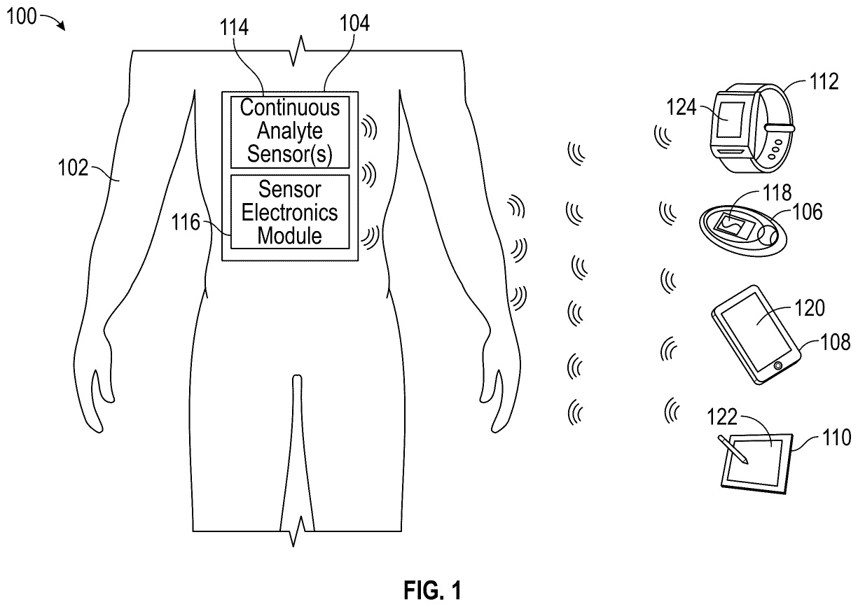

FIG. 1 illustrates an example continuous analyte monitoring system 100, such as a diabetes (or other analyte) management system, that may be used in connection with embodiments of the present disclosure that involve gathering, monitoring, and/or providing information regarding analyte values present in the body of a patient 102, including for example blood glucose values of the patient 102. The continuous analyte monitoring system 100 may continuously monitor one or a plurality of analytes of the patient 102. The continuous analyte monitoring system 100 includes a continuous analyte sensor system 104, and display devices 106, 108, 110, and 112. The continuous analyte sensor system 104 includes one or more continuous analyte sensors 114 and a sensor electronics module 116. The sensor electronics module 116, and the continuous analyte sensor system 104 generally, may be in wired or wireless communication (e.g., directly or indirectly) with one or more of the display devices 106, 108, 110, and 112. In certain embodiments, the continuous analyte sensor system 104 is in direct wired or wireless communication with two or more of the display devices 106, 108, 110, and 112.

Each continuous analyte sensor 114 may include one or more analyte sensors for measuring analytes. For example, the continuous analyte sensor(s) 114 may include a multi-analyte sensor that continuously measures two or more analytes (e.g., glucose, lactate, potassium, ketone, etc.), and/or multiple single analyte sensors, each continuously measuring a single analyte (e.g., where one continuous analyte sensor 114 is used for measuring glucose and then a second continuous analyte sensor 114 used for measuring lactate, etc.). The continuous analyte sensor(s) 114 may include non-invasive devices, minimally-invasive devices, skin-adhered devices, subcutaneous devices, transcutaneous devices, subdermal devices, intradermal devices, transdermal devices, or intravascular devices. The continuous analyte sensor(s) 114 may continuously measure analyte levels of the patient 102 using one or more techniques, such as enzymatic techniques, ion-selective techniques, aptameric techniques, chemical techniques, physical techniques, electrochemical techniques, spectrophotometric techniques, polarimetric techniques, calorimetric techniques, iontophoretic techniques, radiometric techniques, immunochemical techniques, and the like. The continuous analyte sensor(s) 114 may generate one or more signal streams, or an electrical current, indicative of a level (e.g., a concentration) of one or more analytes in the patient 102 over time. The signal stream or current may vary over time as the level of the one or more analytes changes over time.

An analyte may be a substance or chemical constituent in a biological fluid (for example, blood, interstitial fluid, cerebral spinal fluid, lymph fluid, sweat, or urine) that can be analyzed. Analytes can include naturally occurring substances, endogenous substances, exogenous substances, artificial substances, pharmacologic agents, metabolites, electrolytes, ions, blood gasses, minerals, vitamins, proteins, enzymes, or reaction products. Analytes for measurement by the devices and methods may include, but may not be limited to, glucose, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); bicarbonate; biotinidase; biopterin; blood urea nitrogen; c-reactive protein; calcium; carbon dioxide; carnitine; carnosinase; CD4; ceruloplasmin; chenodeoxycholic acid; chloride; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycerol; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; potassium; progesterone; prolactin; prolidase; purine nucleoside phosphorylase; potassium, quinine; reverse tri-iodothyronine (rT3); selenium; serum pancreatic lipase; sissomicin; sodium; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, oxygen parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

Salts, sugar, protein, fat, vitamins, and hormones naturally occurring in blood or interstitial fluids can also constitute analytes in certain implementations. The analyte can be naturally present in the biological fluid, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon, sodium-glucose co-transporter 2 inhibitors (SGLT-2i), glucagon-like peptide 1 (GLP-1) agonists; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, catecholamines (L-DOPA, dopamine, epinephrine, norepinephrine), methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

The sensor electronics module 116 includes electronic circuitry for measuring and processing the signal streams, or an electrical current, from the continuous analyte sensors 114. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, all of which are incorporated herein by reference in their entireties.

The sensor electronics module 116 can be physically connected to the continuous analyte sensors 114 and can be integral with (non-releasably attached to) or releasably attachable to the continuous analyte sensors 114. The sensor electronics module 116 may include hardware (including, but not limited to an electrochemical analog front end, microprocessor, battery, and memory), firmware, or software that enable measurement of levels of analytes via the continuous analyte sensors 114. For example, the sensor electronics module 116 can include an electrochemical analog front end (e.g., a potentiostat, controlled voltage device, galvanostat, controlled current device, coulometer, impedance analyzer, frequency response analyzer, etc.), a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, or a processor. In some embodiments, the sensor electronics module 116 includes a memory and a processor. The memory stores software instructions that are executed by the processor to perform the actions or functions of the continuous analyte sensor system 104 described herein.

The display devices 106, 108, 110, and 112 may display displayable sensor data, including the detected levels of analytes, which may be transmitted by the sensor electronics module 116. In certain embodiments, the sensor electronics module 116 directly transmits sensor data to one, two, three, or more of the display devices 106, 108, 110, and 112 simultaneously or sequentially. The sensor electronics module 116 may transmit raw sensor data that is converted to displayable sensor data via one or more of the display devices 106, 108, 110, and 112. The sensor electronics module 116 may convert raw sensor data to displayable sensor data and transmit the displayable sensor data to one or more of the display devices 106, 108, 110, and 112. Each of the display devices 106, 108, 110, and 112 may include a display such as a touchscreen display 118, 120, 122, and 124 for displaying sensor data to a patient or for receiving inputs from the patient. For example, a graphical user interface (GUI) may be presented to the patient for such purposes. The display devices 106, 108, 110, and 112 may include other types of user interfaces such as a voice user interface instead of, or in addition to, a touchscreen display for communicating sensor data to the patient using the display device or for receiving patient inputs. The display devices 106, 108, 110, and 112 may display or otherwise communicate the sensor data as it is communicated from the sensor electronics module 116 (e.g., in a customized data package that is transmitted to the display devices 106, 108, 110, and 112 based on their respective preferences).

The display devices 106, 108, 110, and/or 112 may include a custom display device specially designed for displaying certain types of displayable sensor data for analyte data received from the sensor electronics module 116. For example, the display device 108 may be a smart phone or a mobile phone using a commercially available operating system (OS) and capable of displaying a graphical representation of the continuous sensor data (e.g., including current and historic data), and the display device 106 may be a continuous analyte monitoring system receiver configured to display graphical representations of the continuous sensor data. The display device 110 may include a tablet, and the display device 112 may include a smart watch. The display devices 106, 108, 110, and 112 may include a desktop or laptop computer (not shown).

Because different display devices provide different user interfaces, content of the data packages (e.g., amount, format, or type of data to be displayed, alarms, and the like) can be customized (e.g., programmed differently by the manufacture or by an end user) for each particular display device. Accordingly, different display devices can be in direct wireless communication with the sensor electronics module 116 (e.g., such as an on-skin sensor electronics module 116 that is physically connected to continuous analyte sensors 114) during a sensor session to enable a plurality of different types or levels of display or functionality for the displayable sensor information. For example, in certain embodiments, the continuous analyte monitoring system 100 may include the display device 112, a smart watch, and the display device 108, a smart phone or mobile phone, wherein both the display device 112 and 108 are in direct wireless communication with the sensor electronics module 116.

The continuous analyte sensor system 104 and/or the display devices 106, 108, 110, and 112 may communicate with each other wirelessly using one of a variety of wireless communication technologies (e.g., Wi-Fi, Bluetooth, Near Field Communication (NFC), NB-IoT, LTE Cat M1, 4G, LTE, 5G, 6G, cellular, etc.). A wireless access point (WAP) may be used to couple one or more of the continuous analyte sensor system 104 or the display devices 106, 108, 110, and 112 to one another. For example, the WAP may provide Wi-Fi, Bluetooth, or cellular connectivity among these devices. NFC may also be used among the devices in the continuous analyte monitoring system 100.

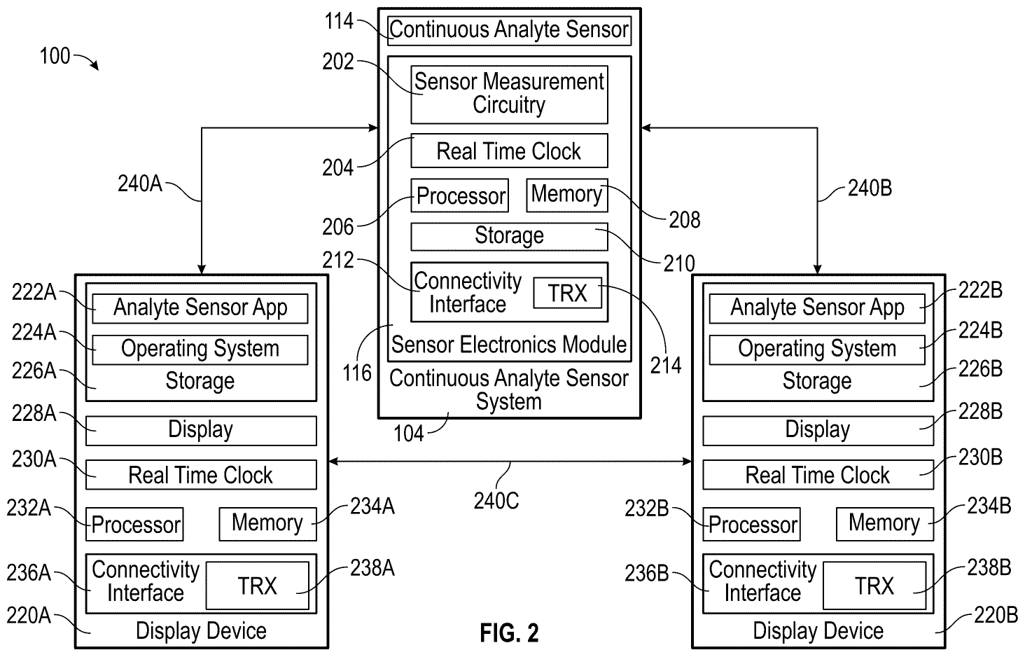

FIG. 2 illustrates a more detailed view of the continuous analyte monitoring system 100 including display devices 220A and 220B (together referred to as “display devices 220”) that are each in direct communication with (e.g., able to directly send and receive signals to/from) the continuous analyte sensor system 104. In certain embodiments, the display devices 220 may be representative of any one of the display devices 106, 108, 110, and 112 of FIG. 1. For example, in certain embodiments, the display device 220a is representative of the display device 108 (e.g., a smart phone or mobile phone) and the display device 220b is representative of the display device 112 (e.g., a smart watch), or vice versa.

The two-way communication paths between the continuous analyte sensor system 104 and the display devices 220A and 220B are shown as communication paths 240A and 240B, respectively. In certain embodiments, the continuous analyte sensor system 104 and the display devices 220 are configured to wirelessly communicate over the communication paths 240A and 240B using low range and/or distance wireless communication protocols. Examples of low range and/or distance wireless communication protocols include Bluetooth and Bluetooth Low Energy (BLE) protocols. In certain embodiments, other short-range wireless communications may include Near Field Communications (NFC), radio frequency identification (RFID) communications, IR (infrared) communications, optical communications. In certain embodiments, wireless communication protocols other than low range and/or distance wireless communication protocols may be used for the communication paths 240A and 240B, such as WiFi Direct. In certain embodiments, the display devices 220 are further configured to wirelessly communicate with each other over a communication path 240C using low range and/or distance wireless communication protocols, such as those communication protocols described above.

The display devices 220 may also be configured to connect to a network (not shown) (e.g., local area network (LAN), wide area network (WAN), the Internet, etc.). For example, the display devices 220 may connect to a network via a wired (e.g., Ethernet) or wireless (e.g., WLAN, wireless WAN, cellular, Mesh network, personal area network (PAN) etc.) interface, to communicate with a network server system coupled to storage (e.g., one or more computer storage systems, cloud-based storage systems and/or services, etc.). The network server system may be configured to receive, collect, and/or monitor information, including analyte data and related information, from the display devices 220. Such information may include input responsive to the analyte data or input (e.g., the patient's analyte measurements and other physiological/behavioral information) received in connection with an analyte monitoring application running on the display devices 220. This information may be stored and processed, such as by an analytics engine capable of performing analytics on the information. Examples of an analyte sensor application that may be executable on the display devices 220A and 220B include analyte sensor applications 222A and 222B, respectively, as further described below.

FIG. 2 also illustrates the components of the continuous analyte sensor system 104 in further detail. As shown, in certain embodiments, the continuous analyte sensor system 104 includes the continuous analyte sensor 114 coupled to the sensor electronics module 116. The sensor electronics module 116 includes sensor measurement circuitry 202 that is coupled to the continuous analyte sensor 114 for processing and managing sensor data. Sensor measurement circuitry 202 may also be coupled to a processor 206. In some embodiments, the processor 206 may perform part or all of the functions of the sensor measurement circuitry 202 for obtaining and processing sensor measurement values from the continuous analyte sensor 114. Software instructions that are executed by the processor 206 to perform the actions or functions of the continuous analyte sensor system 104 described may be stored by a memory 208 communicatively coupled to the processor 206. The processor 206 may also be coupled to storage 210 and real time clock (RTC) 204 for storing and tracking sensor data. In addition, the processor 206 may be further coupled to a connectivity interface 212, which includes a radio unit or transceiver (TRX) 214 for sending sensor data and receiving requests and commands from external devices, such as the display devices 220. As used herein, the term transceiver generally refers to a device or a collection of devices that enable the continuous analyte sensor system 104 to (e.g., wirelessly) transmit and receive data. It is contemplated that, in some embodiments, the sensor measurement circuitry 202 may carry out all the functions of the processor 206, and vice versa.

Storage 210 may be a non-volatile storage for storing instructions, data, etc. For example, storage 210 may store volumes of analyte data collected by continuous analyte sensor 114 for later retrieval and use by continuous analyte monitoring system 100, e.g., for determining alert states after the occurrence of signal loss events. Historical analyte data and/or other sensor data stored in storage 210 for subsequent retrospective processing may be referred to herein as “backfill data.”

The transceiver 214 may be configured with the necessary hardware and wireless communications protocols for enabling wireless communications between continuous analyte sensor system 104 and other devices, such as the display devices 220. For example, as described above, the transceiver 214 may be configured with the necessary hardware and communication protocols to establish a Bluetooth or BLE connection with the display devices 220. As one of ordinary skill in the art appreciates, in such an example, the necessary hardware may include a Bluetooth or BLE security manager and/or other Bluetooth or BLE related hardware/software modules configured for Bluetooth or BLE communications standards. As discussed elsewhere, other short-range protocols may also be used for communication between the display devices 220 and the continuous analyte sensor system 104 such as NFC, RFID, etc. In still other embodiments, the transceiver 214 may be configured with the necessary hardware and wireless communications protocols for long-range wireless cellular communication protocols, such as, GSM, CDMA, LTE, VOLTE, 3G, 4G, 5G communication protocol).

FIG. 2 similarly illustrates the components of display devices 220 in further detail. For clarity, analogous components of display devices 220A and 220B are described together herein. As shown, each display device 220A and 220B includes a connectivity interface 236A or 236B (together referred to as “connectivity interfaces 236”), a processor 232A or 232B (together referred to as “processors 232”), a memory 234A or 234B (together referred to as “memory 234”), a real time clock 230A or 230B (together referred to as “real time clocks 230”), a display 228A or 228B (together referred to as “displays 228”) for presenting a graphical user interface (GUI), and a storage 226A or 226B (together referred to as “storage 226”), respectively. A bus (not shown here) may be used to interconnect the various elements of each display device 220 and transfer data between these elements.

Connectivity interfaces 236 include a transceiver (TRX) 238A or 238B (together referred to as “transceivers 238”), respectively, used for receiving sensor data from the continuous analyte sensor system 104 and for sending requests, instructions, and/or data to the continuous analyte sensor system 104, as well as a network server system. The transceivers 238 are coupled to other elements of display devices 220 via the connectivity interfaces 236, and/or the corresponding bus. The transceivers 238 may include multiple transceiver modules operable on different wireless standards. For example, the transceivers 238 may be configured with one or more communication protocols, such as wireless communication protocol(s) for establishing a wireless communication path with a network, and/or low range wireless communication protocol(s) (e.g., Bluetooth or BLE) for establishing the wireless communication paths 240A or 240B with the continuous analyte sensor system 104 or wireless communication path 240C between the display devices 220A and 220B. Additionally, the connectivity interfaces 236 may in some cases include additional components for controlling radio and/or wired connections, such as baseband and/or Ethernet modems, audio/video codecs, and so on.

In some embodiments, when a standardized communication protocol is used between the display device 220A and/or 220B and the continuous analyte sensor system 104, commercially available transceiver circuits may be utilized that incorporate processing circuitry to handle low level data communication functions such as the management of data encoding, transmission frequencies, handshake protocols, security, and the like. In such embodiments, the processors 232 of the display devices 220, and/or the processor 206 of the continuous analyte sensor system 104, may not need to manage these activities, but instead provide desired data values for transmission, and manage high level functions such as power up or down, set a rate at which messages are transmitted, and the like. Instructions and data values for performing these high-level functions can be provided to the transceiver circuits via a data bus and transfer protocol established by the manufacturer of the transceivers 214 and/or 238. However, in embodiments where a standardized communication protocol is not used between the transceivers 214 and/or 238 (e.g., when non-standardized or modified protocols are used), the processors 206 and/or 232 may be configured to execute instructions associated with proprietary communications protocols (e.g., one or more of the communications protocols described herein) to control and manage their respective transceivers. In addition, when non-standardized or modified protocols are used, customized circuitries may be used to service such protocols.

The processors 232 may include processor sub-modules, including, by way of example, an applications processor that interfaces with and/or controls other elements of display devices 220 (e.g., connectivity interfaces 236, analyte sensor applications 222, displays 228, RTCs 230, memory 234, storage 226, etc.). In certain embodiments, the processors 232 are configured to perform functions related to device management, such as, for example, managing lists of available or previously paired devices, information related to network conditions (e.g., link quality and the like), information related to the timing, type, and/or structure of messaging exchanged between the continuous analyte sensor system 104 and the display devices 220, and so on. The processors 232 may further be configured to receive and process patient input, such as, for example, a patient's biometric information, such as the patient's finger print (e.g., to authorize the patient's access to data or to be used for authorization/encryption of data, including analyte data), as well as analyte data.

The processors 232 may include and/or be coupled to circuitry such as logic circuits, memory, a battery and power circuitry, and other circuitry drivers for periphery components and audio components. The processors 232 and any sub-processors thereof may include logic circuits for receiving, processing, and/or storing data received and/or input to display devices 220, and data to be transmitted or delivered by display devices 220. As described above, the processors 232 may be coupled by a bus to displays 228, connectivity interfaces 236, storage 226, etc. Hence, the processors 232 may receive and process electrical signals generated by these respective elements and thus perform various functions. By way of example, the processors 232 may access stored content from storage 226 and memory 234 at the direction of the analyte sensor applications 222, and process the stored content to be displayed by displays 228. Additionally, the processors 232 may process the stored content for transmission via the connectivity interfaces 236 to the continuous analyte sensor system 104 and/or a server system. The display devices 220 may include other peripheral components not shown in detail in FIG. 2.

In certain embodiments, the memory 234 may include volatile memory, such as random access memory (RAM) for storing data and/or instructions for software programs and applications, such as analyte sensor applications 222. The displays 228 present corresponding GUIs associated with operating systems 224 and/or analyte sensor applications 222. In various embodiments, a patient may interact with the analyte sensor applications 22 via a corresponding GUI presented on displays 228. By way of example, displays 228 may be touchscreen displays that accept touch inputs. Analyte sensor applications 222 may process analyte-related data received by display devices 220 and/or present such data via corresponding displays 228 of the display devices 220. Additionally, the analyte sensor applications 222 may be used to obtain, access, display, control, and/or interface with analyte data and related messaging and processes associated with the continuous analyte sensor system 104 (e.g., and/or any other medical device (e.g., insulin pump or pen) that are communicatively coupled with display devices 220), as is described in further detail herein.

Storage 226 may be a non-volatile storage for storing software programs, instructions, data, etc. For example, storage 226 may store instructions for corresponding analyte sensor application 222 that, when executed using processors 232, for example, receives input (e.g., by a conventional hard/soft key or a touch screen, voice detection, or other input mechanism), and allows a patient to interact with the analyte data and related content via displays 228. In various embodiments, storage 226 may also store patient input data and/or other data collected by display devices 220 (e.g., input from other patients gathered via analyte sensor applications 222). Storage 226 may further be used to store volumes of analyte data received from the continuous analyte sensor system 104 (or any other medical data received from other medical devices (e.g., insulin pump, pen, etc.) for later retrieval and use, e.g., for determining trends and triggering alerts.

As described above, the continuous analyte sensor system 104, in certain embodiments, gathers analyte data from the continuous analyte sensor 114 and transmits the same or a modified version of the collected data to display devices 220. Data points regarding analyte values may be gathered and transmitted over the life of the continuous analyte sensor 114 (e.g., in the range of 1 to 30 days or more). New analyte measurements may be transmitted often enough to adequately monitor glucose levels. In certain embodiments, rather than having the transmission and receiving circuitry of each of continuous analyte sensor system 104 and the display devices 220 continuously communicate, the continuous analyte sensor system 104 and the display devices 220 may regularly and/or periodically establish a communication channel among each other. Thus, in such embodiments, the continuous analyte sensor system 104 may, for example, communicate with one or both display devices 220 at predetermined time intervals. The duration of the predetermined time interval can be selected to be long enough so that the continuous analyte sensor system 104 does not consume too much power by transmitting data more frequently than needed, yet frequent enough to provide substantially real-time sensor information (e.g., measured glucose values or analyte data) to the display devices 220 for output (e.g., via the displays 228) to the patient. While the predetermined time interval is every five minutes in some embodiments, it is appreciated that this time interval can be varied to be any desired length of time. In other embodiments, the transceivers 214 and 238 may be continuously communicating. For example, in certain embodiments, the transceivers 214 and 238 may establish a session or connection therebetween and continue to communicate together until the connection is lost.

The analyte sensor applications 222 may be downloaded, installed, and initially configured/setup on display devices 220. For example, display devices 220 may obtain analyte sensor applications 222 from a network server system, or from another source, such as an application store or the like, via a network. Following installation and setup, analyte sensor applications 222 may be configured to access, process, and/or interface with analyte data (e.g., whether stored on a network server system, locally from storage 226, from the continuous analyte sensor system 104, or any other medical device). By way of example, analyte sensor applications 222 may present menus that include various controls or commands that may be executed in connection with the operation of the continuous analyte sensor system 104, display devices 220, one or more other display devices (e.g., display device 106, 110, etc.), and/or one or more other partner devices, such as an insulin pump. For example, analyte sensor applications 222 may be used to interface with or control other display and/or partner devices, for example, to deliver or make available thereto analyte data, including for example by receiving/sending analyte data directly to the other display and/or partner devices and/or by sending an instruction for the continuous analyte sensor system 104 and the other display and/or partner devices to be connected.

In certain embodiments, after downloading a sensor application 222, as one of the initial steps, the patient may be directed by the sensor application 22 to wirelessly connect the corresponding display device 220 to the patient's continuous analyte sensor system 104, which the patient may have already placed on their body. A wireless communication path 240 between the display device 220 and the continuous analyte sensor system 104 allows the continuous analyte sensor system 104 to transmit analyte measurements to the display device 222 and for the two devices to engage in any of the other interactions described above.

In certain embodiments, sensor data, such as the collected analyte data from the continuous analyte sensor 114, and/or alerts may be sent to a hierarchy of display devices 220. For example, one display device 220 in the continuous analyte monitoring system 100 may be designated as a primary device, or hub, for receiving sensor data, and may control the flow of information and/or alerts to other (secondary, tertiary, etc.) devices in the system, including other display devices. Thus, in some examples, display device 220A may be designated as a primary device, while display device 220B may be designated as a secondary device, or vice versa. In such examples, continuous analyte sensor system 104 may transmit sensor data and/or alerts to the primary device first, before the continuous analyte sensor system 104 and/or primary device transmit the sensor data and/or alerts. In certain embodiments, hierarchical designations for display devices 220 may be based on storage and/or processing capabilities of the display devices 220. In certain embodiments, hierarchical designations for display devices 220 may be swapped upon occurrence of certain events.

### Example Alert States and Associated Events

As noted above, an analyte sensor application (e.g., analyte sensor application 222a or 222b) processes analyte-related data received at a display device (e.g., display device 220a or 220b) from a continuous analyte sensor system (e.g., continuous analyte sensor system 104) in real-time. The analyte-related data typically includes continuous analyte measurements of a patient as taken by a continuous analyte sensor (e.g., continuous analyte sensor 114) of the continuous analyte monitoring system. In addition to processing the continuous analyte measurements for display on the display device, the analyte sensor application processes the analyte measurements to determine and establish an alert state of the display device that corresponds with the analyte measurements. As used herein, an “alert state” may refer to a state of the display device, or the continuous analyte monitoring system generally, that corresponds to an analyte event of the patient or other sensor event (together referred to as “events”). The alert state may be associated with particular settings for notifications (e.g., to be generated and output to the patient) that can be configured and/or programmed by the patient for each analyte event of the patient or sensor event. Accordingly, when the patient is experiencing a particular analyte event at a given time, the display device may determine and automatically enter an alert state that corresponds to the current analyte event and has certain associated notification settings. And while in that alert state, the display device will provide notifications to the patient per the associated notification settings. In certain embodiments, display devices of a continuous analyte monitoring system may be in two or more alert states simultaneously.

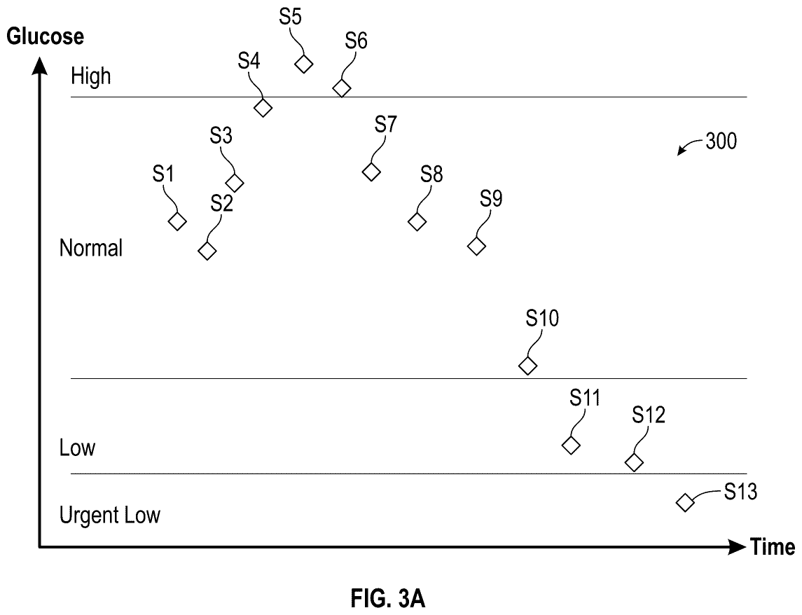

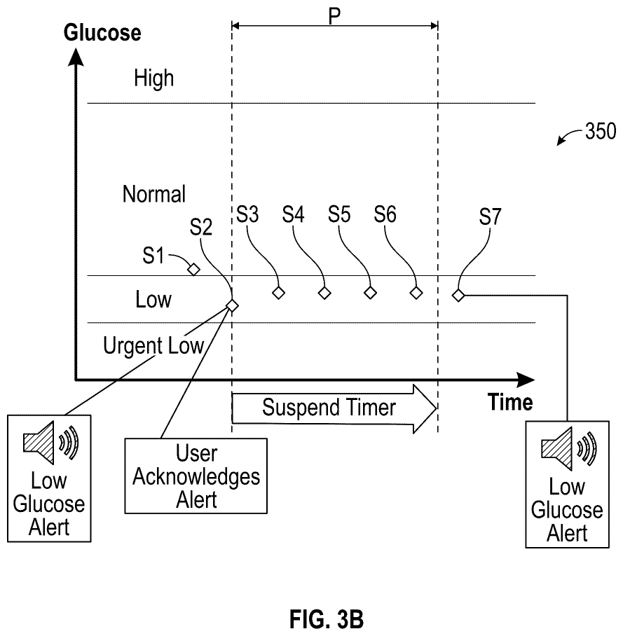

FIG. 3A is a diagram of a time series of glucose measurements 300 illustrating example analyte events for triggering different alert states, in accordance with certain aspects of the present disclosure. As shown in FIG. 3A, four example nominal glucose concentration value ranges are shown: “high,” “normal,” “low,” and “urgent low.” Each of these ranges may be patient-specific, determined by, e.g., a physician or other medical professional, and configured and/or programmed into continuous analyte monitoring system 100 via, e.g., an analyte sensor application 222. When a patient's glucose concentration values fall within these nominal ranges, a display device 220, and/or the continuous analyte monitoring system 100 generally, may enter a corresponding alert state. For example, for time points S1-S4 and S7-S10 of FIG. 3A, the display device 220 may enter a “normal” alert state; for time points S5 and S6, the display device 220 may enter a “high” alert state; for time points S11 and S12, the display device 220 may enter a “low” alert state; and for time point S13, the display device 220 may enter a “urgent low” alert state.

In addition to the above, a difference between contiguous glucose concentration values of a patient may correspond to an alert state of the display device 220 and/or continuous analyte monitoring system 100. For example, if a rate of change in contiguous glucose concentration values meets a predetermined threshold value, the display device 220 may enter a corresponding alert state. Examples of such analyte events in FIG. 3A include time points S2-S5, which represent a “fast rising” analyte event, and time points S6-S8 and S9-S11, which represent a “fast falling” analyte event or “urgent low soon” analyte event. These analyte events may have corresponding “fast rising,” “fast falling,” and “urgent low soon” alert states. In certain embodiments, a “fast falling” analyte event is based on one or measurements of a rate of change of glucose concentration values. In certain embodiments, an “urgent low soon” analyte event is based on one or more algorithms or models for predicting glucose concentration values of the patient at a future time point based on glucose measurements 300. Such future time point may be 5 minutes, 10 minutes, 20 minutes, 30 minutes, or more in the future relative to the most recent measurement or the current time. In certain embodiments, the “urgent low soon” analyte event occurs when a predicted future glucose measurement falls below a target threshold value.

In other examples, a sensor issue or operating error may correspond to an alert state of the display device 220 and/or continuous analyte monitoring system 100. For example, the inability of the sensor 114 to collect analyte measurements may cause the display device 220 to enter a corresponding “sensor issue” alert state. Examples of scenarios that may cause the sensor 114 to be unable to collect analyte measurements include: biological responses of the patient that encapsulate one or more components of the sensor 114 and prevent analytes from reaching the sensor 114 for measurement; sensor material degradation; physical damage to one or more components of the sensor 114; utilization of the sensor 114 outside of operating temperature ranges; electronics failure of one or more components of one or more components of the sensor 114; battery or power failure; end-of-life of the sensor 114; a sensor firmware failure; and the like.

In certain embodiments, the display device 220 and/or continuous analyte monitoring system 100 may include one or more modes for an alert state. Each “mode” may depend on the occurrence of an event while the display device 220 is in the corresponding alert state, such as the generation/output of a notification related to the alert state and/or the acknowledgement of a notification by the patient. Accordingly, the display device 220 may transition between two or more modes while in a single alert state. In certain embodiments, an alert state may include the following modes: an “idle” mode, where no alert is generated and output to the patient; a “notification” mode, where a triggering event has occurred and a notification is generated for output to the patient; and an “acknowledged” mode, where an alert was output and acknowledged by the patient, and the triggering event is still valid or occurring. In certain embodiments, a “notification” mode or “acknowledged” mode may automatically transition to an “idle” mode when the triggering event is no longer present or valid. In certain embodiments, an alert state may have a default mode, which may be set based on patient notification preferences and/or a risk level associated with the corresponding alert state. For example, a “normal” alert state may have a default “idle” mode, while a “high,” “low,” and/or “urgent low” alert state may have a default “notification” mode. In such examples, the occurrence of an event while the display device 220 is in a given alert state may cause a shift from the default mode to another mode.

In certain embodiments, an alert state may also be associated with one or more timers. Such timers may govern the amount of time alerts are suppressed by the display device 220 after the occurrence of an event, such as the onset of an analyte event, the generation/output of a notification related to an alert state, and/or the acknowledgement of a notification by the patient. Accordingly, such timers may be herein referred to as “alert timers,” and an alert state may be associated with one or more types of alert timers. Examples of alert timers may include: “repeat” timers, which prevent the same notification type from being generated and output to the patient within a certain amount of time; “suspension” timers, which suspend some or all other notification types from being generated and output to the patient within a certain amount of time; and “cue” timers, which suppress or postpone one or more notification types until an event, such as an analyte event, has occurred for a threshold minimum amount of time. In certain embodiments, an alert timer may suspend one or more types of notifications from being output to a patient, while allowing one or more other types of notifications to be output. For example, a suspension timer may suspend notification types associated with the same alert state or improved alert states (e.g., corresponding with improved analyte events), while allowing notifications types associated with worsened alert states (e.g., corresponding with worsened analyte events).

FIG. 3B is another diagram of a time series of glucose measurements 350 illustrating the triggering of an alert state and an associated timer, in accordance with certain aspects of the present disclosure. As shown in FIG. 3B, the glucose measurements 400 include glucose measurements taken at seven time points, S1-S7. At time point S1, a glucose measurement of the patient is within a “normal” nominal glucose concentration value range, and so the display device 220 may enter a corresponding “normal” alert state. In such a state, without any events triggering the generation/output of a notification, the display device 220 may be operating in a default “idle” mode for the normal alert state.

Then, at time point S2, the glucose level drops into a “low” nominal glucose concentration value range, and the continuous analyte monitoring system 100 enters a corresponding “low” glucose alert state. In this example, the low glucose concentration value, and thus the operation of the display device 220 in the low glucose alert state, automatically triggers the display device 220 to operate in a default notification mode and output a corresponding notification to the patient. Shortly after the notification is output in this example, the notification is acknowledged by the patient, which in turn triggers the initiation of a timer according to one or more settings associated with the low glucose alert state and operation of the display device 220 in an acknowledged mode. The timer may include a “repeat” or a “suspension” timer that prevents the output of certain further notifications for a time period (e.g., duration) P, which corresponds to operation of the display device 220 in the acknowledged mode.

In FIG. 3B, four glucose measurements are taken at time points S3-S6 during the time period P, which are all in the “low” range. As a result of there being no worsening circumstances during the time period P, no further notifications are output to the patient until the glucose measurement at time point S7, which occurs after the time period P. And, because the patient's glucose levels are still within the “low” range, another notification corresponding to the low glucose alert state is output.

### Example Signal Loss Scenarios

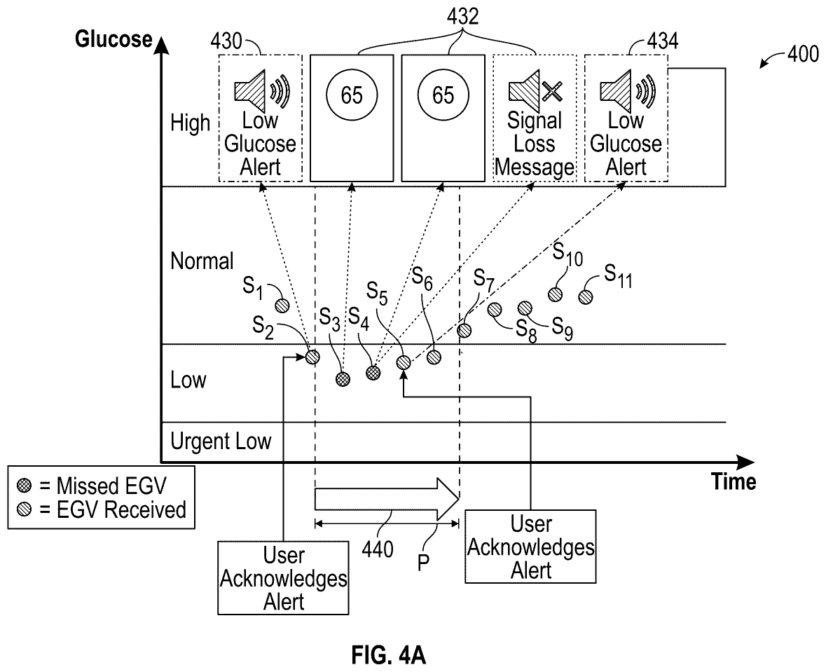

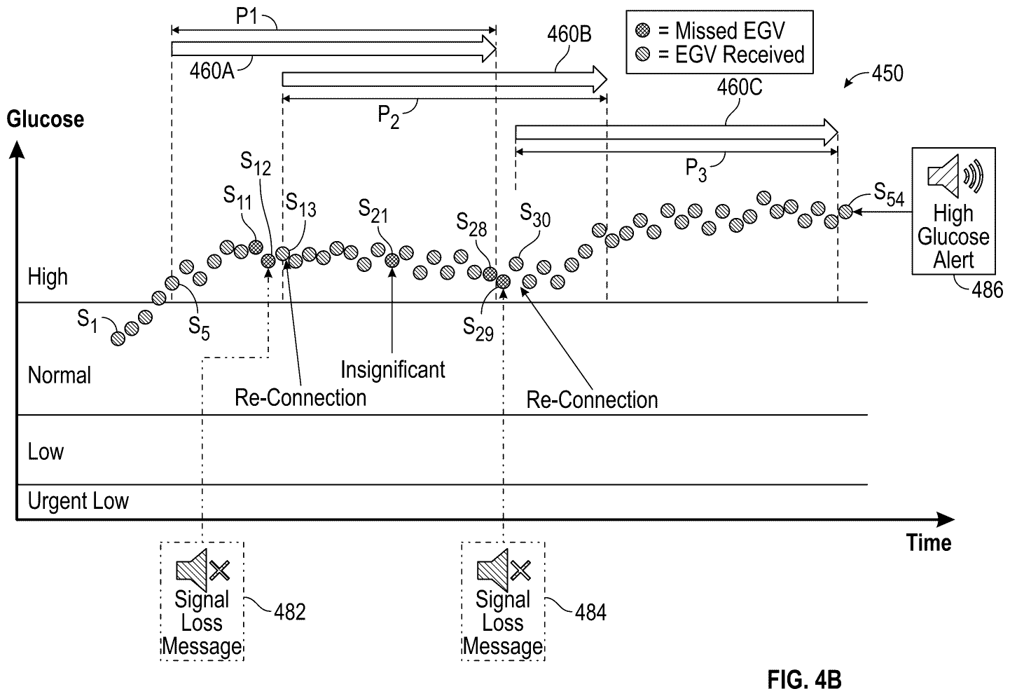

FIG. 4A illustrates an example signal loss scenario that may result in redundant alerts being generated and output to a patient, in accordance with certain aspects of the present disclosure. In particular, FIG. 4A depicts a time series of glucose measurements 400 and associated events or actions as carried out by conventional continuous glucose monitoring techniques.

As shown in FIG. 4A, the glucose measurements 400 include glucose measurements taken at eleven time points, S1-S11. At time point S1, a glucose measurement of the patient is within a “normal” nominal glucose concentration value range, and so the continuous analyte monitoring system enters a corresponding “normal” glucose alert state. In such a state, without any events triggering the generation/output of a notification, the continuous analyte monitoring system may be operating in a default “idle” mode for the normal glucose alert state.

At time point S2, the patient's glucose measurements 400 fall into a “low” nominal glucose concentration value range, which triggers the continuous glucose monitoring system to enter a default notification mode of a low glucose alert state. A low glucose alert 430 indicating the low glucose levels of the patient is generated and output to the patient via an associated display device. In this scenario, the patient sees and acknowledges the alert 430 via, e.g., the display device. The acknowledgement of the alert 430 by the patient triggers the start of an alert timer 440, which suspends additional notifications for a time period P, e.g., 20 minutes. As described elsewhere herein, the alert timer 440 may be one of a plurality of types of alert timers governing how long additional notifications are to be suppressed by the continuous glucose monitoring system following acknowledgement by the patient. Examples of alert timer types include repeat timers, suspension timers, and cue timers, as described above.

In the current scenario, while the alert timer 440 is in progress, a signal loss event occurs, wherein the connection between the patient's display device and the analyte sensor system of the continuous glucose monitoring system is disrupted. The signal loss event may be caused by interference from other devices, the patient setting the display device down and walking a far distance away from the display device, the patient placing the analyte sensor system or display device in a particular position or location that blocks the transmission/reception of the information packets (e.g., the patient's pocket), power loss or rebooting of the display device, and the like. In FIG. 4A, the signal loss event is a significant signal loss event that persists while glucose measurements of the patient are taken by the analyte sensor system at two time points S3 and S4, as indicated by hatching of the data plots at time points S3 and S4. Here, the term “significant” refers to a signal event occurring for a threshold minimum amount of time, e.g., spanning two glucose measurements, as shown in FIG. 4A. During this signal loss event, the patient's display device does not receive the glucose measurements taken by the analyte sensor system.

In conventional analyte monitoring systems, upon occurrence of a significant signal loss event, all prior glucose alerts and their associated patient acknowledgements and timers on the display device are dismissed. Thus, in the scenario depicted in FIG. 4A, the low glucose alert 430 and its associated timer 440 are dismissed during the significant signal loss event spanning time points S3 and S4. Accordingly, all alert states of the continuous glucose monitoring system are set to an “idle” mode.

In some examples, upon occurrence of a significant signal loss event, the display device may display a “silent” notification 432, without an accompanying audible alert, as shown in FIG. 4A. In such examples, the patient may not become aware of the signal loss event due to the silent nature of the silent notification 432. Where a signal loss event is not significant, or does not occur for the threshold minimum amount of time, the display device may not provide any notifications to the patient indicating the occurrence of the signal loss event.

At about time point S5, the connection between the patient's display device and the analyte sensor system is re-established. At this point, the patient's glucose measurements 400 are still in the low nominal glucose concentration value range. Because the low glucose alert 430 and its associated timer 440 were previously dismissed, the continuous glucose monitoring system re-enters the default notification mode, and a new low glucose alert 434 is generated and output to the patient to be acknowledged. In examples where the patient is not aware of the occurrence of the signal loss event, the patient may become confused as to why another low glucose notification was output so quickly. And, even where the patient is aware of the signal loss between the patient's display device and the analyte sensor system, if multiple successive signal loss events were to occur, the patient may become fatigued and/or annoyed by the redundant notifications. Accordingly, the repetition or redundancy of notifications by conventional analyte monitoring techniques and systems, as caused by signal loss events may lead to alert fatigue and confusion of the patient in certain scenarios. The generation and presentation of redundant alerts on the display device also presents an unnecessary processing burden on the display device.

FIG. 4B illustrates another example signal loss scenario that may result in significant delays in alerts being generated and output to a patient, in accordance with certain aspects of the present disclosure. In particular, FIG. 4B depicts a time series of glucose measurements 450 and associated events or actions as carried out by conventional continuous glucose monitoring techniques.

As shown in FIG. 4B, the glucose measurements 450 include glucose measurements taken at fifty-four time points, S1-S54. At time point S1, a glucose measurement of the patient is within a “normal” nominal glucose concentration value range, and so the continuous analyte monitoring system enters a corresponding “normal” glucose alert state. In such a state, without any events triggering the generation/output of a notification, the continuous analyte monitoring system may be operating in a default “idle” mode for the normal glucose alert state.

At about time point S5, the patient's glucose measurements 400 rise into a “high” nominal glucose concentration value range, which triggers the continuous glucose monitoring system to enter a default notification mode of a high glucose alert state. In the example of FIG. 4B, the notification mode for the high glucose alert state is associated with a cue timer. Accordingly, upon entry into the high alert state, a first cue timer 460A is initiated, which suppresses or postpones the generation and output of a notification indicating the high glucose levels of the patient until the high glucose levels have persisted for a threshold minimum period of time, e.g., 120 minutes, as indicated by time period P1 in FIG. 4B.

In the current scenario, while the alert timer 460A is in progress, a first signal loss event occurs while glucose measurements of the patient are taken by the analyte sensor system at time points S11 and S12, as indicated by hatching of the data plots at time points S11 and S12. This first signal loss event is “significant,” and thus, all prior glucose alerts and their associated patient acknowledgements and timers on the display device are dismissed. Accordingly, in the scenario depicted in FIG. 4B, the timer 460A is dismissed during the signal loss event spanning time points S11 and S12, and all alert states of the continuous glucose monitoring system are set to an “idle” mode.

Because the first signal loss event is a significant signal loss event, the display device displays a silent notification 482 for the patient, without an accompanying audible alert. In this example, the patient may not become aware of the notification 482 due to its silent nature, and further, may not yet become aware of their high glucose levels since a high glucose alert has not yet been output.

At about time point S13, the connection between the patient's display device and the analyte sensor system is re-established. At this point, the patient's glucose measurements 450 are still in the high nominal glucose concentration value range. Because the timer 460A previously associated with the high glucose alert state was dismissed during the signal loss event, the continuous glucose monitoring system re-enters the notification mode for the high glucose alert state, and another cue timer 460B with a time period P2, e.g., 120 minutes, is started.

A second signal loss event occurs while a glucose measurement of the patient is taken by the analyte sensor system at time point S21, as indicated by hatching of the data plot at time point S21. However, because this second signal loss event is not significant (e.g., is short in duration and does not occur for a set minimum time threshold), the timer 460B is not dismissed, and no notifications are output regarding the signal loss event.

While glucose measurements of the patient are taken by the analyte sensor system at time points S28 and S29, a third signal loss event occurs, as indicated by hatching of the data plots at time points S28 and S29. Unlike the second signal loss event, this third signal loss event is significant, and so the timer 460B is dismissed and another silent notification 484 is displayed for the patient. When the connection between the patient's display device and the analyte sensor system is re-established at about time point S30, the patient's glucose levels are still in the high nominal glucose concentration value range. Consequently, the continuous glucose monitoring system once again re-enters the notification mode for the high glucose alert state, and another cue timer 460C with a time period P3, e.g., 120 minutes, is started.

No further signal loss events occur during the time period P3, and so a high glucose alert 486 is finally generated and output at the conclusion of P3, at about time point S54. Due to the occurrence of the two significant signal loss events in the scenario of FIG. 4B, the time between the onset of the patient's high glucose levels and the output of the high glucose alert 486 is significantly extended, resulting in a significantly delayed notification to the patient. Thus, using conventional analyte monitoring techniques and systems, where one or more signal loss events occur, a patient may not be made aware of an analyte event until long after its onset.

### Alert State Backfill Following Signal Loss

Described below are several techniques that may be employed by one or more display devices of a continuous analyte monitoring system for alert state recovery and/or adjustment following signal loss events between an analyte sensor system and a display device. The following techniques avoid scenarios that can cause redundant alerts from being generated and output to a patient, which can lead to alert fatigue of the patient, and/or scenarios that can cause significant delays in the output of alerts, which can lead to the delay in treatment of analyte events of the patient. In certain embodiments, the following techniques also avoid unnecessary processing of large volumes of data, which can reduce the processing burden on a display device and/or other devices of the continuous analyte monitoring system when recovering and updating a current alert state of the system.

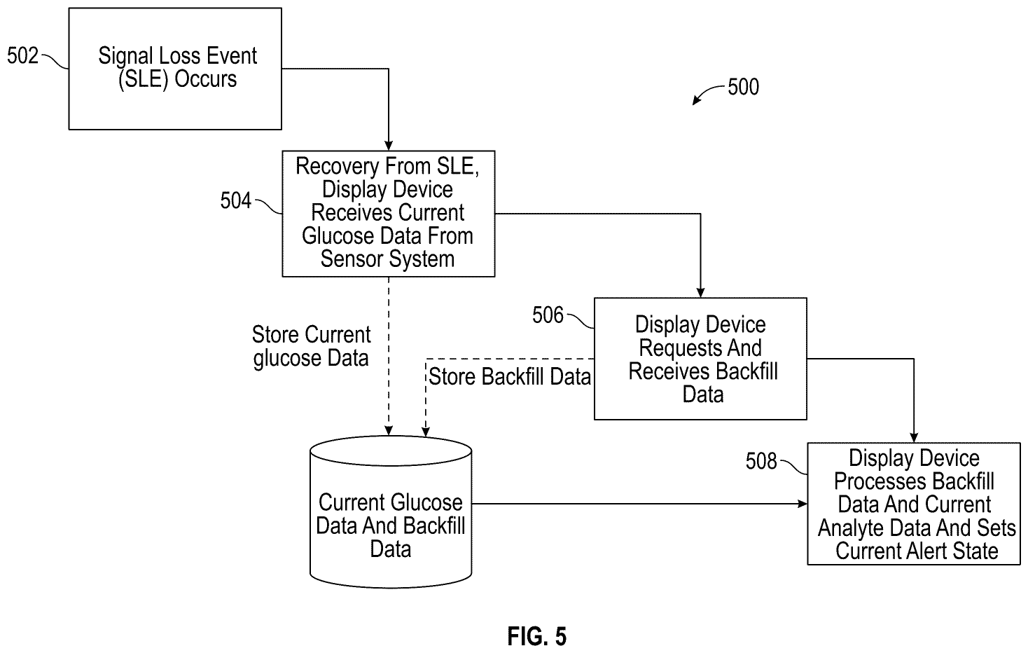

In a first approach, or mode of operation, as described with reference to FIG. 5, backfill sensor data from a duration of a signal loss event is received by a recovered display device from an analyte sensor system, and the backfill data is retrospectively processed and analyzed to adjust a current alert state and mode of the continuous analyte monitoring system, as well as associated timers and other settings.

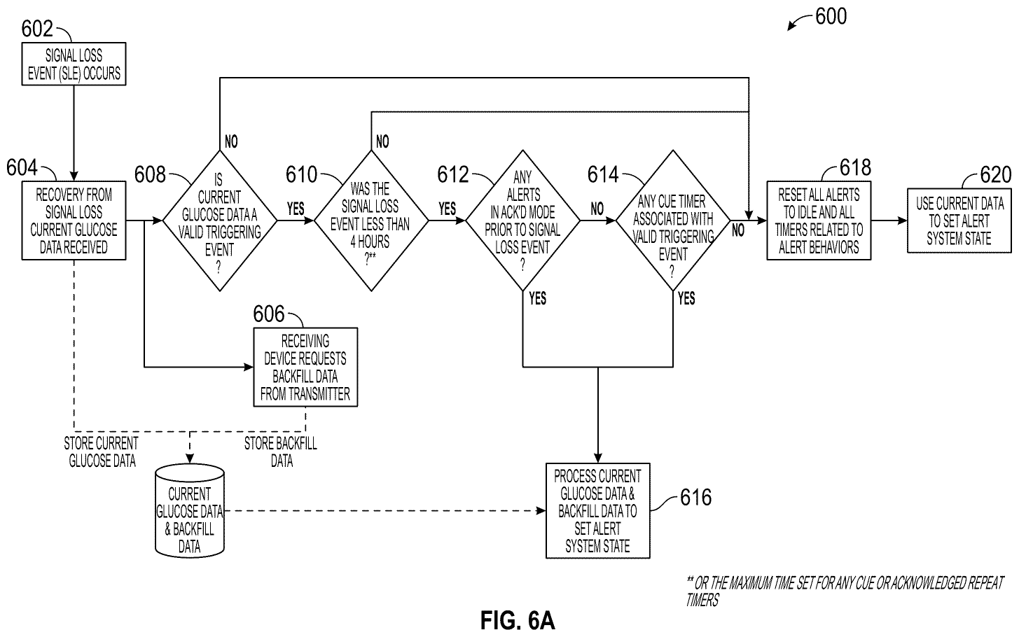

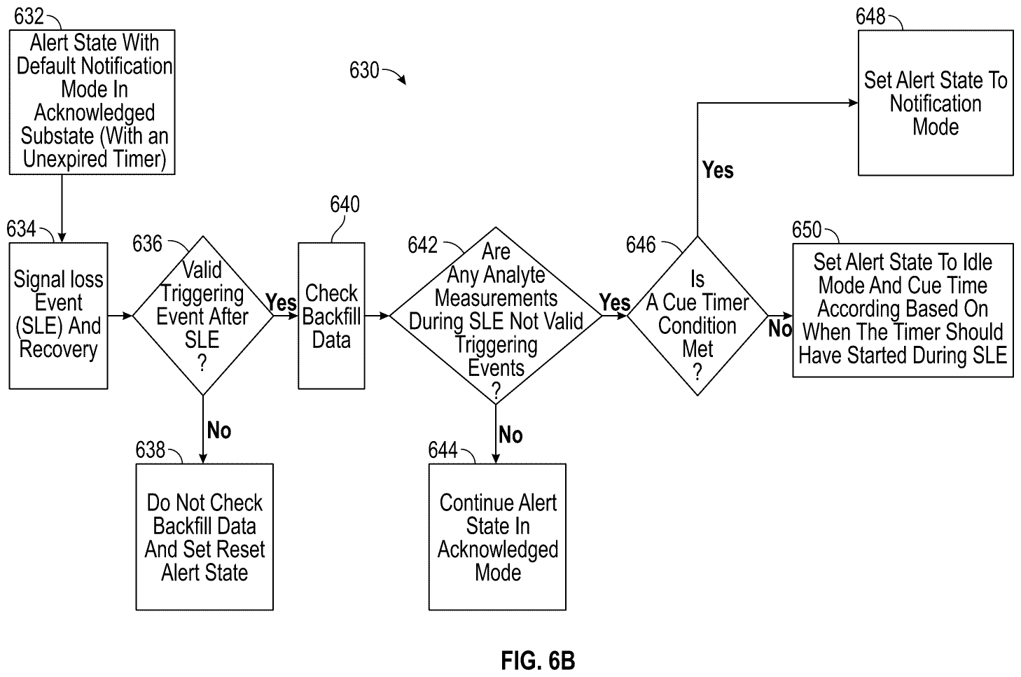

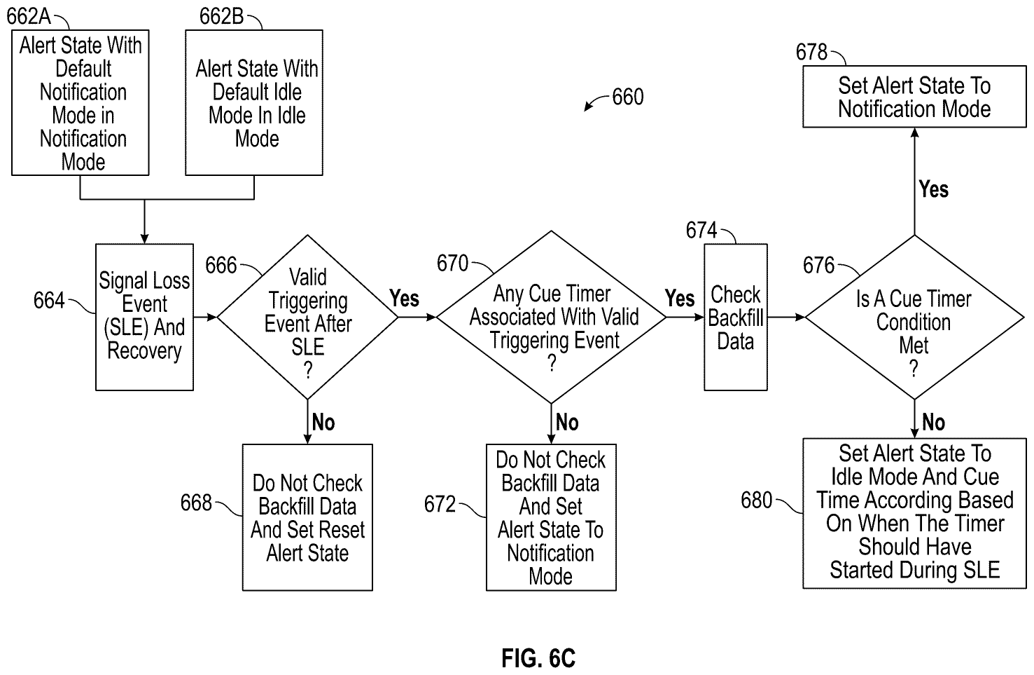

In a second approach, or mode of operation, as described with reference to FIGS. 6A-6C, the recovered display device implements a series of rules to conditionally process the backfill sensor data from the duration of the signal loss event, thereby reducing the computing power needed to achieve the same or similar results as the first approach above.

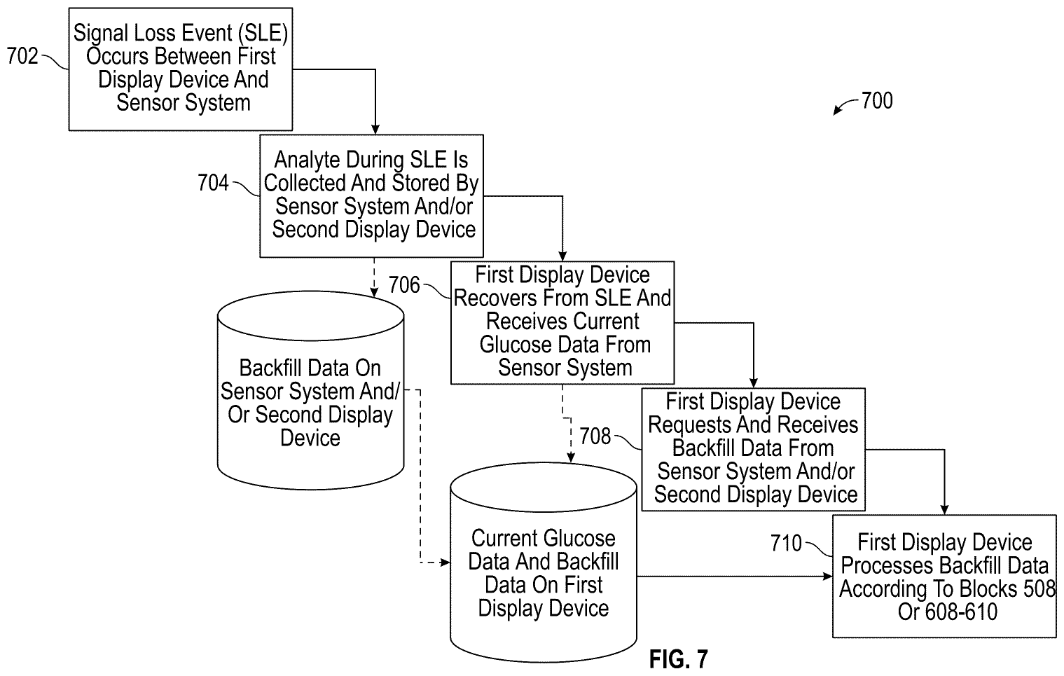

In a third approach, or mode of operation, as described with reference to FIG. 7, signal loss event backfilling is implemented by a continuous analyte monitoring system including an analyte sensor system in direct communication with a first display device and a second display device. In response to a signal loss event between the first display device and the analyte sensor system, the second display device and/or the analyte sensor system may cache the backfill data, and then transmit the cached backfill data to the first display device upon recovery. The backfill data may then be processed, or conditionally processed, according to the first and second approaches.

These three approaches, as well as other techniques, are described in further detail below.

Turning to FIG. 5, a flow diagram of an example method 500 of the first approach for recovery of prior alert states and associated conditions and/or settings after a signal loss event is depicted, in accordance with certain aspects of the present disclosure. In particular embodiments, the method 500 may be performed by one or more components of the continuous analyte monitoring system 100. Accordingly, operations of method 500 will be described herein with reference to continuous analyte monitoring system 100.

At block 502, a signal loss event occurs between a continuous analyte sensor system 104 and at least one display device 220 of the continuous analyte monitoring system 100. Prior to the signal loss event, the display device 220 may be operating in at least one of a plurality of alert states and at least one of a plurality of corresponding modes. Examples of alert states include the “high,” “normal,” “low,” “urgent low,” “fast rising,” “fast falling,” “urgent low soon,” and “sensor issue” alert states described above. Examples of modes include the “idle,” “notification,” and “acknowledged” modes described above. Further, one or more timers associated with the alert states and/or alert substrates may be running prior to the signal loss event. Examples of timer types include “repeat” timers, “suspension” timers, and “cue” timers, as described above.

To cause the signal loss event, the wireless (or wired) connection between the continuous analyte sensor system 104 and the display device 220 is disrupted, thereby preventing the display device 220 from receiving information packets including measured analyte levels of a patient from the continuous analyte sensor system 104. Generally, the signal loss event may be instigated by one or more of a variety of scenarios, such as interference from other devices, the patient setting the display device down and walking a far distance away from the display device, the patient placing the analyte sensor system or display device in a particular position or location that blocks the transmission and/or reception of the information packets, power loss or rebooting of the display device, and the like.

During the signal loss event, the continuous analyte sensor system 104 continues to collect and process analyte data of the patient, including measured analyte levels. The analyte data collected during the signal loss event is stored by the continuous analyte sensor system 104 in, e.g., storage 210, for later retrieval and use by the display device 220. In certain embodiments, the analyte data collected during the signal loss event is only temporarily stored by the continuous analyte sensor system 104.

In certain embodiments, the display device 220 identifies the occurrence of the signal loss event based upon the non-receipt of information packets from the continuous analyte sensor system 104. For example, in certain embodiments, identification of the occurrence of the signal loss event is based on a determination of a deviation from an expected cadence (e.g., frequency) of receiving packets from the continuous analyte sensor system 104 during normal operating conditions. In such embodiments, the display device 220 may generate and output a notification indicating the occurrence of the signal loss event to the patient. In certain embodiments, the notification is a silent notification including a visual alert (e.g., on display 118, 120, 122, and 124) and no audible or tactile alert. In certain embodiments, the notification includes an audible and/or tactile alert to indicate the occurrence of the signal loss event.

At block 504, the continuous analyte monitoring system 100 recovers from the signal loss event, and the connection between the continuous analyte sensor system 104 and the display device 220 is re-established. Upon recovery, information packets including current analyte data (e.g., current measured analyte levels) of the patient are received at the display device 220, and the current analyte data is stored on the display device, e.g., in storage 226. Based at least in part on the receipt of the information packets from the continuous analyte sensor system 104, the display device 220 determines that the signal loss event has ended.

In certain embodiments, the display device 220 determines whether the signal loss event at block 504 is a significant signal loss event prior to moving to block 506. For example, in certain embodiments, the display device 220 determines whether the signal loss event occurred for a minimum amount of time, e.g., spanning two glucose measurements or more, prior to performing block 506. In such embodiments, if the signal loss event does not occur for the threshold time limit, the method does not progress to block 506.

At block 506, the display device 220 requests backfill data from the continuous analyte sensor system 104, including the analyte data collected and stored by the continuous analyte sensor system 104 during the signal loss event (e.g., historical analyte data). In response, the continuous analyte sensor system 104 transmits the backfill data to the display device 220, which stores the backfill data in, e.g., storage 226. In certain embodiments, the backfill data is thereafter removed from (e.g., deleted) storage in the continuous analyte sensor system 104.

At block 508, the display device 220 processes the backfill data and the current analyte data received from the continuous analyte sensor system 104 to determine and set a current alert state of the continuous analyte monitoring system 100 and any associated conditions or settings, including associated timers. In certain embodiments, setting the current alert state of the continuous analyte monitoring system 100 may include at least one of: starting one or more new timers for a full or partial time period(s) corresponding with the timer(s); continuing one or more timers initiated prior to the signal loss event; cancelling one or more timers initiated prior to the signal loss event; changing an alert state and/or a mode set prior to the signal loss event; or continuing an alert state and/or a mode set prior to the signal loss event. Generally, the setting of the current alert state may be based on one or more events (e.g., analyte events, sensor events, etc.) indicated by the backfill data and/or the current analyte data.

In certain embodiments, processing the backfill data at block 508 is automatically executed by the display device 220 upon receiving the backfill data from the continuous analyte sensor system 104 at block 506. In certain embodiments, the processing of the backfill data includes processing and analyzing, or evaluating, all of the analyte data collected by the continuous analyte sensor system 104 during the signal loss event. In certain embodiments, the processing of the backfill data includes processing and analyzing a portion of the analyte data collected by the continuous analyte sensor system 104 during the signal loss event.

In certain embodiments, processing the backfill data at block 508 includes forward processing of the backfill data, wherein backfill data is processed in chronological order starting with analyte data collected following the signal loss event. In certain embodiments, processing the backfill data at block 508 includes backward processing of the backfill data, wherein backfill data is processed in reverse chronological order starting with the most recent analyte data collected during the signal loss event.

While the above approach avoids redundant alerts from being generated and output to a patient after a signal loss event, and/or avoids alerts from being delayed as a result of erroneously restarted timers, this approach requires the storage and processing of all backfill data collected during the signal loss event to determine the current state of a continuous analyte monitoring system. Thus, this first approach may only be performed by certain devices having sufficient storage and processing capabilities. In the following approach, however, a set of rules is implemented to conditionally process the backfill data, which reduces the computing power needed to achieve the same or similar results. As such, this second approach may be performed by a greater variety of devices with varying storage and/or processing capabilities.

Turning to FIG. 6A, a flow diagram of an example method 600 of the second approach for recovery of prior alert states and associated conditions and/or settings after a signal loss event is depicted, in accordance with certain aspects of the present disclosure. In particular embodiments, the method 600 may be performed by one or more components of the continuous analyte monitoring system 100. Accordingly, operations of method 600 will be described herein with reference to continuous analyte monitoring system 100.

At block 602, a signal loss event occurs between the continuous analyte sensor system 104 and at least one display device 220 of the continuous analyte monitoring system 100. The signal loss event may be caused by a wireless (or wired) connection between the continuous analyte sensor system 104 and the display device 220 being disrupted, thereby preventing the display device from receiving information packets, including measured analyte levels of a patient, from the continuous analyte sensor system 104. The signal loss event may be caused by one or more of a variety of scenarios, such as interference from other devices, the patient setting the display device down and walking a far distance away from the display device, the patient placing the analyte sensor system or display device in a particular position or location that blocks the transmission and/or reception of the information packets, power loss or rebooting of the display device, and the like.

During the signal loss event, the continuous analyte sensor system 104 continues to collect and process analyte data of the patient, including measured analyte levels. The analyte data collected during the signal loss event is stored by the continuous analyte sensor system 104 in, e.g., storage 210 for later retrieval and use by the display device 220. In certain embodiments, the analyte data collected during the signal loss event is only temporarily stored by the continuous analyte sensor system 104.

In certain embodiments, the display device 220 identifies the occurrence of the signal loss event based upon the non-receipt of information packets from the continuous analyte sensor system 104. In such embodiments, the display device 220 may generate and output a notification indicating the occurrence of the signal loss event to the patient. In certain embodiments, the notification is a silent notification including a visual alert (e.g., on display 118, 120, 122, and 124) and no audible or tactile alert. In certain embodiments, the notification includes an audible and/or tactile alert to indicate the occurrence of the signal loss event.

At block 604, the continuous analyte monitoring system 100 recovers from the signal loss event, and the connection between the continuous analyte sensor system 104 and the display device 220 is re-established. Upon recovery, information packets including current analyte data (e.g., current measured analyte levels) of the patient are received at the display device 220, and the current analyte data is stored on the display device, e.g., in storage 226. Based at least in part on the receipt of the information packets from the continuous analyte sensor system 104, the display device 220 determines that the signal loss event has ended.

In certain embodiments, the display device 220 determines whether the signal loss event at block 604 is a significant signal loss event prior to moving to block 606. For example, in certain embodiments, the display device 220 determines whether the signal loss event occurred for a threshold minimum amount of time, e.g., an amount of time spanning two glucose measurements or more, prior to performing block 606. In such embodiments, if the signal loss event does not occur for the threshold time limit, the method does not progress to block 606.

At block 606, the display device 220 requests backfill data from the continuous analyte sensor system 104, including the analyte data collected and stored by the continuous analyte sensor system 104 during the signal loss event. In response, the continuous analyte sensor system 104 transmits the backfill data to the display device 220, which stores the backfill data in, e.g., storage 226. In certain embodiments, the backfill data is thereafter removed from storage in the continuous analyte sensor system 104.

At block 608, the display device 220 processes the current analyte data (e.g., current measure analyte levels) of the patient to determine whether the patient is currently experiencing an analyte state corresponding to an alert state of the continuous analyte monitoring system 100 that would normally trigger a notification. For example, the display device 220 may process the most recent one or more analyte measurements received from the continuous analyte sensor system 104 to determine whether the patient's current analyte levels are within one or more nominal concentration ranges corresponding to an alert state that would normally trigger a notification, are following a trend (e.g., rate of change, etc.) corresponding to an alert state that would normally trigger a notification, and/or meet any other set criteria that would normally trigger a notification. Such an event may be referred to as a “valid triggering event.”

If, at block 608, the display device 220 identifies a valid triggering event, the method proceeds to block 610. If, at block 608, the display device 220 determines that the patient is not currently experiencing a valid triggering event, the method proceeds to block 618.

At block 610, the display device 220 determines if the signal loss event at block 602 had a duration less than a threshold maximum amount of time (e.g., a maximum time limit). In certain embodiments, the threshold amount of time is based on the time period of one or more alert timers of the continuous analyte monitoring system 100. In such embodiments, the threshold amount of time may be equivalent to, or greater than, the maximum time period set for all alert timers of the continuous analyte monitoring system 100, including repeat timers, suspension timers, and cue timers, as described above. For example, in a continuous analyte monitoring system 100 having a patient-specific maximum cue timer of four hours upon onset of a high analyte alert state for sending a notification, the threshold amount of time of block 610 may be four hours. In this example, evaluating the duration of the signal loss event against the threshold amount of time may indicate whether any previously set timers would have expired during the signal loss event, which may suggest that the display device 220 can “skip” its processing of the backfill data and efficiently and accurately reset the current alert state based on the current analyte data.

If, at block 610, the display device 220 determines that the signal loss event occurred for less than the threshold amount of time, the method proceeds to block 612. If, at block 610, the display device 220 determines that the signal loss event had a duration greater than the threshold amount of time, and therefore, that any previously set timer would have lapsed during the signal loss event, the method proceeds to block 618.

At block 612, the display device 220 determines whether the continuous analyte monitoring system 100 was operating in an acknowledged mode upon onset of the signal loss event. For example, the display device 220 processes data collected by the display device 220 prior to the signal loss event, including historical analyte data, display device operating data, and patient input data, to determine whether a notification had been output and thereafter acknowledged by a patient, such as to cause the continuous analyte monitoring system 100 to operate in an acknowledged mode prior to the signal loss event.

If, at block 612, the display device 220 determines that the continuous analyte monitoring system 100 was operating in an acknowledged mode upon onset of the signal loss event, the method proceeds to block 616. If, at block 612, the display device 220 determines that the continuous analyte monitoring system 100 was not operating in an acknowledged mode upon onset of the signal loss event, the method proceeds to block 614.

At block 614, the display device 220 determines whether any cue timers are associated with the valid triggering event. For example, the display device 220 processes data collected by the display device 220 after the signal loss event, including current analyte data, display device operating data, and/or patient input data, to determine whether the continuous analyte monitoring system 100 should be operating in a notification mode if the conditions of the cue timer were met upon recovering from the signal loss event.

If, at block 614, the display device 220 determines that a cue timer is associated with the valid triggering event, the method proceeds to block 616. If, at block 614, the display device 220 determines that a cue timer is not associated with the valid triggering event, the method proceeds to block 618.

At block 616, the display device 220 processes the backfill data and the current analyte data received from the continuous analyte sensor system 104 to determine and set a current alert state of the continuous analyte system 100. Again, the processing of the backfill data and the current analyte data at block 616 is performed only if the display device 220 determines that the continuous analyte monitoring system 100 was operating in an acknowledged mode upon onset of the signal loss event, or that a cue timer was initiated and running upon onset of the signal loss event. By performing these conditional checks, the continuous analyte monitoring system 100 can reduce the processing burden for recovering the alert state of the system by entirely avoiding the processing of the backfill data and instead resetting alert states, modes, and/or timers at block 618, as described below.

In certain embodiments, setting the current alert state of the continuous analyte monitoring system 100 may include at least one of: starting one or more new timers for a full or partial time period(s) corresponding with the timer(s); continuing one or more timers initiated prior to the signal loss event; cancelling one or more timers initiated prior to the signal loss event; changing an alert state and/or an mode set prior to the signal loss event; or continuing an alert state and/or an mode set prior to the signal loss event. Generally, the setting of the current alert state may be based on one or more events (e.g., analyte events, sensor events, etc.) indicated by the backfill data and/or the current analyte data.

In certain embodiments, setting the current alert state of the continuous analyte monitoring system 100 may include at least one of: starting one or more new timers for a full or partial time period(s) corresponding with the timer(s); continuing one or more timers initiated prior to the signal loss event; cancelling one or more timers initiated prior to the signal loss event; changing an alert state and/or an mode set prior to the signal loss event; or continuing an alert state and/or an mode set prior to the signal loss event. Generally, the setting of the current alert state may be based on one or more events (e.g., analyte events, sensor events, etc.) indicated by the backfill data and/or the current analyte data.

In certain embodiments, processing the backfill data at block 616 includes forward processing of the backfill data, wherein backfill data is processed in chronological order starting with analyte data collected following onset of the signal loss event. In certain embodiments, processing the backfill data at block 616 includes backward processing of the backfill data, wherein backfill data is processed in reverse chronological order starting with the most recent analyte data collected during the signal loss event.

Turning to block 618, if the display device 220 determines that the signal loss event at block 602 did not occur for less than the threshold amount of time, or that the patient is not currently experiencing an analyte state corresponding to an alert state that would normally trigger a notification, the display device 220 resets all alert states (e.g., to the “normal” alert state) and further resets all modes (e.g., to the “idle” mode) and all timers associated with the alert states and/or modes. Thereafter, at block 620, the display device 220 sets the current alert state for the continuous analyte monitoring system 100 based on the processed current analyte data. For example, in certain embodiments, the current alert state is based on the processed current analyte data without the backfill data.

FIGS. 6B and 6C illustrate flow diagrams depicting example decision logic implemented by a continuous analyte monitoring system during various signal loss scenarios according to the alert state recovery method 600 of FIG. 6A, in accordance with certain aspects of the present disclosure. Operations performed in FIGS. 6B and 6C will be described herein with reference to one or more components of the continuous analyte monitoring system 100.

Turning to FIG. 6B, a first signal loss scenario 630 is depicted. In this scenario, a display device 220 of the continuous analyte monitoring system 100 is operating in an acknowledged mode of an alert state prior to a signal loss event that lasted less than a set threshold amount of time. In this scenario, a patient has recently received and acknowledged a notification output by the display device 220 indicating the alert state, and so the display device 220 is operating in an acknowledged mode with an unexpired suspension timer prior to the signal loss event. These operating conditions are indicated at block 632.

At block 634, a signal loss event occurs between the display device 220 and a corresponding continuous analyte sensor system 104 of the continuous analyte monitoring system 100, and communication between the display device 220 and the continuous analyte sensor system 104 is thereafter restored. Upon reconnection, current analyte data and backfill data from the continuous analyte sensor system 104 is received by the display device 220 (e.g., corresponding to blocks 604 and 606 of method 600). Further, in this scenario, the display device 220 determines that the signal loss event endured for less than the threshold amount of time, as described in block 610 above.

At block 636, the display device 220 evaluates the current analyte data to determine the occurrence of any valid triggering events immediately following the signal loss event. As used herein, a “valid triggering event” refers to an event that relates to the alert state and mode operating prior to a signal loss event. For example, a “valid triggering event” may be of the same type of analyte event or sensor event that caused the display device 220 to enter the alert state and/or mode operating prior the signal loss event, or caused the starting of an associated timer. Again, the alert state may include, for example, a “high,” “normal,” “low,” “urgent low,” “urgent low soon,” “fast rising,” or “fast falling” alert state (or the like), while the mode may include an “acknowledged,” “idle,” or “notification” mode (or the like).

If, based on the current analyte data, the display device 220 determines that there are no valid triggering events immediately following the signal loss event, then, at block 638, the display device 220 does not process or analyze the backfill data, and instead resets the current alert state based on the current analyte data. For example, if the first analyte measurement upon signal recovery between the display device 220 and the continuous analyte sensor system 104 is not a valid triggering event, the display device 220 may then process and analyze the current analyte data at block 638 to determine and set the current alert state for the display device 220.

If, based on the current analyte data, the display device 220 determines that a valid triggering event occurs immediately following the signal loss event, then at block 640, the display device 220 does process and analyze the backfill data received from the continuous analyte sensor system 104 to determine and set the current alert state for the display device 220. For example, if the first analyte measurement upon signal recovery between the display device 220 and the continuous analyte sensor system 104 is a valid triggering event, the display device 220 may then process and analyze the backfill data at block 640.

At block 642, the display device 220 evaluates the backfill data to determine if any analyte measurements collected during the signal loss event were not valid triggering events. Again, as used herein, a “valid triggering event” refers to an event that relates to the alert state and mode operating prior to a signal loss event.

If, based on the backfill data, the display device 220 determines that all analyte measurements were valid triggering events during the signal loss event, then at block 644, the display device 220 remains in the alert state and corresponding acknowledged mode operating prior to the signal loss event. Further, any timers, such as the described suspension timer, are resumed according to an amount of time that would have been remaining for the timer(s) had the signal loss event not occurred. When following the logic at block 644, a new notification will not be generated and output to the patient until previously pending timers expire and another triggering event occurs.

If, based on the backfill data, the display device 220 determines that at least one analyte measurement was not a valid triggering event during the signal loss event, then at block 646, the display device 220 determines whether the analyte measurements during the signal loss event would have normally triggered a cue timer had the signal loss event not occurred, and whether the conditions of the cue timer would have been met by the first analyte measurement upon signal recovery (e.g., an event associated with the cue timer occurred for the full time period of the cue timer).

If, based on the backfill data, the display device 220 determines that a cue timer would have been triggered, and that the conditions of the cue timer would have been met by the first analyte measurement after signal recovery, then at block 648, the current alert state is set to an alert state corresponding to the event during the signal loss that triggered the cue timer, and the mode is set to a notification mode for that alert state. Accordingly, at block 648, a notification is generated and transmitted to the patient based on the current alert state.

If, based on the backfill data, the display device 220 determines that a cue timer would have been triggered and that the conditions of the cue timer would not have been met by the first analyte measurement after signal recovery, or that a cue timer would not have been triggered until the first analyte measurement after signal recovery, then at block 650, the current alert state is set to an alert state corresponding to the event during the signal loss that triggered the cue timer, or the event indicated by the first analyte measurement after signal recovery. Further, a cue timer is initiated according to an amount of time that would have been remaining for the cue timer had the signal loss event not occurred, and the mode is set to an idle mode for the determined current alert state. When following the logic at block 650, a new notification will not be generated and output to the patient until the set cue timer expires or another triggering event occurs.

Turning now to FIG. 6C, a second signal loss scenario 660 is depicted. In this scenario, a display device 220 of the continuous analyte monitoring system 100 may be operating in one of at least two alert states prior to a signal loss event: a first alert state in a notification mode, as shown in block 662A; and a second alert state in an idle mode, as shown in block 662B. In the current scenario 660, the mode of the alert state in block 662A may transition to the idle mode during the signal loss event, which may be the same mode/alert state prior to the signal loss event in block 662B. And, with reference to block 662B, any initiated timers are unexpired prior to signal loss.

At block 664, a signal loss event occurs between the display device 220 and a corresponding continuous analyte sensor system 104 of the continuous analyte monitoring system 100, and communication between the display device 220 and the continuous analyte sensor system 104 is thereafter restored. Upon reconnection, current analyte data and backfill data from the continuous analyte sensor system 104 is received by the display device 220 (e.g., corresponding to blocks 604 and 606 of method 600). Further, in this scenario, the display device 220 determines that the signal loss event endured for less than the threshold amount of time, as described in block 610 of the method 600.

At block 666, the display device 220 evaluates the current analyte data to determine the occurrence of any valid triggering events immediately following the signal loss event. Again, as used herein, a “valid triggering event” refers to an event that relates to the alert state and mode operating prior to a signal loss event. For example, a “valid triggering event” may be of the same type of analyte event or sensor event that caused the display device 220 to enter the alert state and/or mode operating prior the signal loss event, or caused the starting of an associated timer. Again, the alert state may include, for example, a “high,” “normal,” “low,” “urgent low,” “urgent low soon,” “fast rising,” or “fast falling” alert state (or the like), while the mode may include an “acknowledged,” “idle,” or “notification” mode (or the like).

If, based on the current analyte data, the display device 220 determines that there are no valid triggering events immediately following the signal loss event, then at block 668, the display device 220 does not process or analyze the backfill data, and instead resets the current alert state based on the current analyte data. For example, if the first analyte measurement upon signal recovery between the display device 220 and the continuous analyte sensor system 104 is not a valid triggering event, the display device 220 may then process and analyze the current analyte data at block 668 to determine and set the current alert state for the display device 220.

If, based on the current analyte data, the display device 220 determines that a valid triggering event occurs immediately following the signal loss event, then at block 670, the display device 220 determines whether any cue timers are associated with the valid triggering event. For example, the display device 220 processes data collected by the display device 220 after the signal loss event, including current analyte data, display device operating data, and patient input data, to determine whether the continuous analyte monitoring system 100 should be operating in a notification mode having an associated cue timer upon recovering from the signal loss event.

If, at block 670, the display device 220 determines there are no cue timers associated with the valid triggering event, then at block 672, the display device 220 does not process or analyze the backfill data, and instead sets the current alert state based on the current analyte data and sets the corresponding mode to a “notification” mode to alert the patient based on the current alert state. For example, if the first analyte measurement upon signal recovery between the display device 220 and the continuous analyte sensor system 104 is a valid triggering event and there is no associated cue timer, the display device 220 then sets the current alert state for the display device 220 in a notification mode for the alert state associated with the valid triggering event.

If, at block 670, the display device 220 does determine that there is a cue timer associated with the valid triggering event, then at block 674, the display device 220 processes and analyzes the backfill data received from the continuous analyte sensor system 104 to determine and set the current alert state for the display device 220. For example, if the first analyte measurement upon signal recovery between the display device 220 and the continuous analyte sensor system 104 is a valid triggering event and there is an associated cue timer, the display device 220 may then process and analyze the backfill data at block 674

At block 676, the display device 220 then determines whether the conditions of the associated cue timer (e.g., the event associated with the cue timer occurred for the full time period of the cue timer) would have been met by the first analyte measurement upon signal recovery.

If, based on the backfill data, the display device 220 determines at block 676 that the conditions of the associated cue timer would have been met by the first analyte measurement after signal recovery, then at block 678, the current alert state is set to an alert state corresponding to the event during the signal loss that triggered the cue timer, and the mode is set to a notification mode for that alert state. Accordingly, at block 678, a notification is generated and transmitted to the patient based on the current alert state.

If, based on the backfill data, the display device 220 determines at block 676 that the conditions of the associated cue timer would not have been met by the first analyte measurement after signal recovery, then at block 680, the current alert state is set to an alert state corresponding to the analyte or sensor event during signal loss event that triggered the cue timer, or the event indicated by the first analyte measurement after signal recovery. Further, a cue timer is initiated according to an amount of time that would have been remaining for the associated cue timer had the signal loss event not occurred, and the mode is set to an idle mode for the determined current alert state. When following the logic at block 680, a new notification will not be generated and output to the patient until the set cue timer expires or another triggering event occurs.

FIG. 7 illustrates a flow diagram depicting an example method 700 of the third approach for recovery of prior alert states and associated conditions and/or settings after a signal loss event, in accordance with certain aspects of the present disclosure. In this third approach, signal loss event backfilling is implemented by a continuous analyte monitoring system including a continuous analyte sensor system in direct communication with two display devices. In response to a signal loss event between one display device and the sensor system, the other display device and/or the sensor system caches the backfill data, and then transmits the cached backfill data to the one display device upon recovery.

In particular embodiments, the method 700 may be performed by continuous analyte monitoring system 100. Accordingly, operations of method 700 will be described herein with reference to continuous analyte monitoring system 100, and particularly, continuous analyte sensor system 104 and display devices 220A and 220B, according to certain embodiments. In certain embodiments, the display devices 220A and 220B may be representative of a smart phone and smart watch, respectively or vice versa, according to certain embodiments.

At block 702, a signal loss event occurs between the continuous analyte sensor system 104 and the first display device 220A. During the signal loss event, a wireless connection between the continuous analyte sensor system 104 and the first display device 220A is disrupted, thereby preventing the first display device 220A from receiving information packets including measured analyte levels of a patient from the continuous analyte sensor system 104. The signal loss event may be caused by one or more of a variety of scenarios, as noted above. During the signal loss event, the continuous analyte sensor system 104 continues to collect and process analyte data of the patient, including measured analyte levels.

In certain embodiments, the first display device 220A identifies the occurrence of the signal loss event based upon the non-receipt of information packets from the continuous analyte sensor system 104, and generates and outputs a notification indicating the occurrence of the signal loss event to the patient. In certain embodiments, the notification is a silent notification including a visual alert on a display of the first display device 220A, but no audible or tactile alert. In certain embodiments, the notification includes an audible and/or tactile alert to indicate the occurrence of the signal loss event.

At block 704, the analyte data collected during the signal loss event is temporarily stored by the continuous analyte sensor system 104, and/or transmitted to and stored by the second display device 220B, for later retrieval and use by the first display device 220A. In certain embodiments, at least a portion of the analyte data collected during the signal loss event is stored by the continuous analyte sensor system 104 in, e.g., storage 210. For example, in certain embodiments, all of the analyte data collected during the signal loss event is stored by the continuous analyte sensor system 104. In certain embodiments, a first portion of the analyte data collected during the signal loss event is stored by the continuous analyte sensor system 104, and a second portion of the analyte data collected during the signal loss event is transmitted to and stored by the second display device 220B, e.g., in storage 226. In still other embodiments, all of the analyte data collected during the signal loss event is transmitted to and stored by the second display device 220B.

In certain embodiments, one or more checks (e.g., evaluations) may be performed to determine where (e.g., on what device) the analyte data collected during the signal loss event is to be stored at block 704. Generally, such checks may include determining whether the collected analyte data can be stored on the continuous analyte sensor system 104, and/or whether the collected analyte data can be transmitted and stored on the second display device 220B. For example, in certain embodiments, the one or more checks may include determining the storage and processing capabilities of the continuous analyte sensor system 104 and/or the second display device 220B, and, based on the determined storage and processing capabilities of the continuous analyte sensor system 104 and/or the second display device 220B, storing the collected analyte data at the continuous analyte sensor system 104 and/or the second display device 220B.

In certain embodiments, based on the determined storage and processing capabilities of the second display device 220B, receiver device designations of the display devices 220A and 220B may be swapped during signal loss events. For example, the first display device 220A may be initially designated as a primary device and the second display device 220B designated as a secondary device as described above, but in response to the signal loss event, the second display device 220B may be re-designated as the primary device and the first display device 220A may be designated as the secondary device. In such scenarios, the second display device 220B may maintain alert state and mode information, associated timers, and the like, and may propagate this information to the first display device 220A when communications are available again between the first display device 220A and the continuous analyte sensor system 104.

At block 706, the continuous analyte monitoring system 100 recovers from the signal loss event, and the connection between the continuous analyte sensor system 104 and the first display device 220A is re-established. Upon recovery, information packets including current analyte data (e.g., current measured analyte levels) of the patient are received at the first display device 220A, and the current analyte data is stored on the display device, e.g., in storage 226. Based at least in part on the receipt of the information packets from the continuous analyte sensor system 104, the first display device 220A determines the termination of the signal loss event.

At block 708, the first display device 220A requests backfill data from the continuous analyte sensor system 104 and/or the second display device 220B, including the analyte data collected and stored by the continuous analyte sensor system 104 during the signal loss event. In response, the continuous analyte sensor system 104 and/or the second display device 220B transmits the backfill data to the first display device 220A, which stores the backfill data in, e.g., storage 226. In certain embodiments, the backfill data is thereafter removed from (e.g., deleted) storage in the continuous analyte sensor system 104 and/or second display device 220B.

At block 710, the first display device 220A then processes the backfill data and the current analyte data received from the continuous analyte sensor system 104 and/or the second display device 220B to set the current analyte state of the continuous analyte monitoring system 100. The processing of the backfill data and the current analyte data may be according to either block 508 of the method 500 or method 600, depending on a selected approach for the first display device 220A. For example, the first display device 220A may process all of the backfill data according to method 500, or the display device 220A may perform one or more conditional checks to determine whether to process the backfill data or just reset the alert state, mode, and/or associated timers.

Alert state backfill (i.e., recovery) following signal loss events may be performed by the continuous analyte monitoring system 100 according to one or more of the approaches described above with references to FIGS. 5-8. Thus, in certain embodiments, prior to or during the performance of methods 500, 600, and/or 700, the continuous analyte monitoring system 100 may select between the three approaches for alert state backfill.

Generally, selection between the approaches may be based, at least in part, on a number and type of display devices 220 utilized with the continuous analyte monitoring system 100. Accordingly, in certain embodiments, an approach for a device of the continuous analyte monitoring system 100 may be selected based on the storage and processing capabilities of one or more display devices 200 of the continuous analyte monitoring system 100. For example, where the continuous analyte monitoring system 100 includes a continuous analyte monitoring receiver (e.g., display device 106) with limiting processing capability, and the continuous analyte monitoring receiver experiences a signal loss event, the continuous analyte monitoring system 100 may perform alert state backfill according to the second approach described above due to the limited processing power of the receiver. In another example, where the continuous analyte monitoring system 100 includes a smart phone (e.g., display device 108) running an analyte monitoring application, and the smart phone experiences a signal loss event, the continuous analyte monitoring system 100 may perform alert state backfill according to the first approach described above due to the increased processing power of the smart phone.

In certain embodiments, different display devices 220 of the continuous analyte monitoring system 100 may perform alert state recovery utilizing different approaches. For example, the first display device 220A may perform alert state recovery according to the first approach, while the second display device 220B may perform alert state recovery according to the approach.

In certain embodiments, following a signal loss event, two or more display devices 220 may synchronize their corresponding alert states with one another. For example, where a second display device 220B operating according to the second approach experiences a signal loss event, the second device 220B may adopt or synchronize its alert state with that of a first display device 220A operating according to the first approach. In this example, because the first display device 220A operates according to the first approach, the first display device 220A therefore has evaluated all of the backfill data collected and transmitted by the continuous analyte sensor system 104.

In certain embodiments, one or more blocks of the methods 500, 600, and/or 700 may be delegated to different devices of the continuous analyte monitoring system 100, such that performance of the methods 500, 600, and/or 700 is coordinated between two or more devices. For example, where multiple display devices 220 are utilized with the continuous analyte monitoring system 100, two or more display devices 220 may coordinate with one another to concertedly (e.g., collaboratively) perform one or more operations of the methods 500, 600, and/or 700. In a particular example, where a signal loss event is experienced by a smart watch that is being used in parallel with a smart phone, the first approach described above may be performed to recover the alert state of the smart watch, wherein blocks 506 and 508 are performed by the smart phone instead of the smart watch. In this example, the alert state of the smart watch may be updated after the signal loss event based on the processing of the backfill data by the smart phone. Thus, operations of the methods 500, 600, and/or 700 may be delegated to different devices (e.g., display devices 220 and/or analyte sensors system 104) of the continuous analyte monitoring system 100 for collective performance of such methods.

In an illustrative example, the method 700 can be performed by continuous analyte monitoring system 100 including the continuous analyte sensor system 104, a first display device 220A comprising a smart phone, and a second display device 220B comprising a smart watch. In this example, both the smart phone and the smart watch directly receive analyte data from the continuous analyte sensor system 104 prior to performance of the method 700. At block 702, a signal loss event occurs between the continuous analyte sensor system 104 and the smart phone. At block 704, the analyte data collected during the signal loss event (e.g., backfill data) is cached by the continuous analyte sensor system 104 and/or the smart watch. At block 706, the smart phone recovers from the signal loss event, and subsequently requests backfill data from the continuous analyte sensor system 104 and/or smart watch at block 708. Then, at block 710, the smart phone retrospectively analyzes the backfill data to set an alert state and/or mode of the smart phone, or resets an alert state and/or mode of the smart phone without processing of the backfill data, according to the methods 500 and/or 600.

In another illustrative example, the method 700 can be performed by continuous analyte monitoring system 100 including the continuous analyte sensor system 104, a first display device 220A comprising a smart watch, and a second display device 220B comprising a smart phone. Similar to the previous example, both the smart phone and the smart watch directly receive analyte data from the continuous analyte sensor system 104 prior to performance of the method 700. At block 702, a signal loss event occurs between the continuous analyte sensor system 104 and the smart watch. At block 704, the analyte data collected during the signal loss event (e.g., backfill data) is cached by the continuous analyte sensor system 104 and/or the smart phone. At block 706, the smart watch recovers from the signal loss event, and subsequently requests backfill data from the continuous analyte sensor system 104 and/or smart phone at block 708. Then, at block 710, the smart watch retrospectively analyzes the backfill data to set an alert state and/or mode of the smart watch, or resets an alert state and/or mode of the smart watch without processing of the backfill data, according to the methods 500 and/or 600.

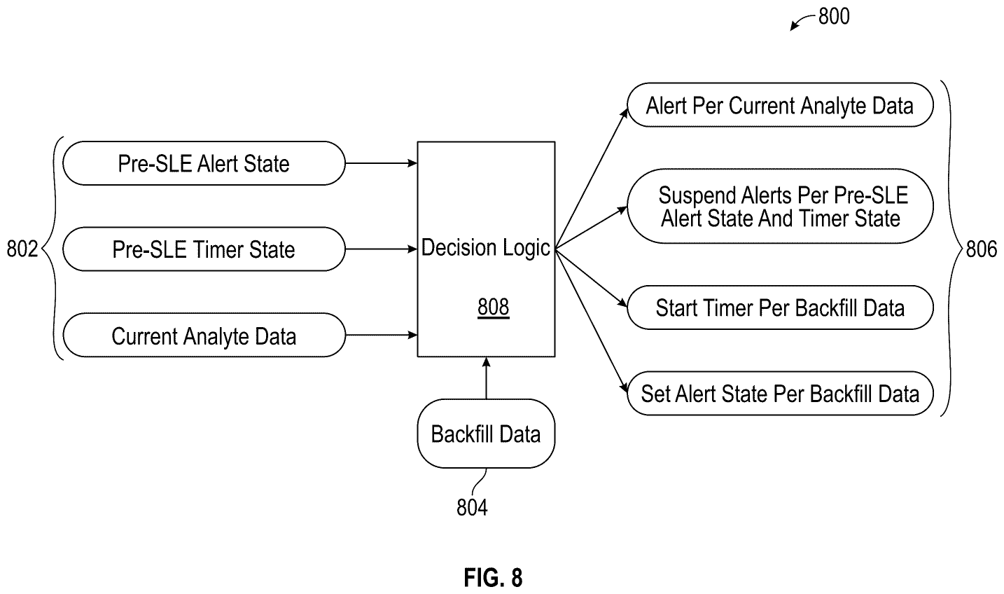

FIG. 8 illustrates a flow diagram of system logic 800 depicting example inputs 802 utilized during the retrospective analysis of backfill data 804 as performed according to the methods described here, as well as the corresponding output actions 806 of such analysis.

Turning to FIG. 8, various items of information may be considered by a decision logic 808 of, e.g., analyte sensor applications 222 or other engines or devices performing the current methods, when processing backfill data to determine and set a current analyte state of a continuous analyte monitoring system. Examples of such inputs 802 include: an alert state of the corresponding display device prior to (e.g., upon) onset of a signal loss event; an mode of the corresponding display device prior to onset of a signal loss event; a state (e.g., status or time elapsed) of any timers associated with the alert state or mode of the corresponding display device prior to onset of a signal loss event; and the current analyte data. Based on the processing of these inputs 802 and the backfill data 804, the decision logic 808 may then perform one or more output actions 806, which can include: starting one or more new timers for a full or partial time period(s) corresponding with the timer(s); continuing one or more timers initiated prior to the signal loss event; cancelling one or more timers initiated prior to the signal loss event; changing an alert state and/or an mode set prior to the signal loss event; continuing an alert state and/or an mode set prior to the signal loss event; and generating and outputting a notification to the patient based on a determined alert state, mode, timer, analyte event, etc.

In summary, aspects of the present disclosure provide techniques for recovering and/or updating an alert state of a continuous analyte monitoring system after the occurrence of a signal loss event between a display device and a corresponding analyte sensor system. In certain embodiments, these techniques may include the retrospective analysis of backfill data that is cached at the analyte sensor system or another device during a signal loss event, and is later transmitted to the display device upon signal recovery. Based on the retrospective analysis of the backfill data, the display device may adjust or update a current alert state and any associated conditions or settings. The retrospective analysis of backfill data cached at the analyte sensor system avoids unnecessary repetition of alerts after signal loss, and enables the subsequent recovery of prior alert states and associated acknowledgements and timers. In certain embodiments, the display device may perform one or more conditional checks (i.e., evaluations) after the signal loss event to determine whether retrospective analysis of the backfill data is needed, or whether a full reset of the alert state based on current analyte data is appropriate. In such embodiments, the performance of conditional checks prior to analysis of the backfill data may relieve the bulk of the processing burden of the display device during the aforementioned alert state recovery.

The methods disclosed herein include one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The term “continuous,” as used herein, is a broad term, and is used in its ordinary sense, and can mean continuous, semi-continuous, continual, periodic, intermittent, regular, etc.

The terms “continuous analyte sensor,” “continuous multi-analyte sensor,” “continuous glucose sensor,” and “continuous lactate sensor,” as used herein, are broad terms, and are used in their ordinary sense, and refer without limitation to a device that continuously measures a concentration of an analyte or calibrates the device (e.g., by continuously adjusting or determining the sensor's sensitivity and background), for example, at time intervals ranging from fractions of a second up to, e.g., 1, 2, or 5 minutes, or longer.

The term “sensor data,” as used herein, is a broad term, and is used in its ordinary sense, and refers without limitation to any data associated with a sensor, such as a continuous analyte or continuous multi-analyte sensor. Sensor data includes a raw data stream, or simply data stream, of analog or digital signal directly related to a measured analyte from an analyte sensor (or other signal generated from another sensor), as well as calibrated or filtered raw data.

The terms “sensor data point” and “data point” refer generally to a digital representation of sensor data at a particular time. The terms broadly encompass a plurality of time spaced data points from a sensor, such as a continuous analyte sensor, which includes individual measurements taken at time intervals ranging from fractions of a second up to, e.g., 1, 2, or 5 minutes or longer. In another example, the sensor data includes an integrated digital value representative of one or more data points averaged over a time period. Sensor data may include calibrated data, smoothed data, filtered data, transformed data, or any other data associated with a sensor.

Although certain embodiments herein are described with reference to management of diabetes, diabetes management is only an example of one application for which the present systems and methods may be utilized. The systems and methods described herein can also be used for managing one or more other diseases or conditions, which may or may not include diabetes. For example, the systems and methods described herein can be utilized for managing kidney disease, liver disease, and other types of diseases or conditions.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘including’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “including as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

### EXAMPLE EMBODIMENTS

Embodiment 1: A method for providing improved data accuracy associated with a display device of an analyte monitoring system, comprising: re-establishing a wireless communication signal between a display device and an analyte sensor system after a signal loss event; upon re-establishing the wireless communication signal, receiving, by the display device, current analyte data of a patient from the analyte sensor system; requesting, by the display device, backfill data from the analyte sensor system, the backfill data comprising historical analyte data of the patient collected by the analyte sensor system during the signal loss event; receiving, by the display device, the backfill data from the analyte sensor system; and processing, by the display device, at least the current analyte data to determine a current alert state of the display device.
Embodiment 2: The method of Embodiment 1, wherein processing at least the current analyte data comprises determining whether the patient is currently experiencing an analyte state corresponding to an alert state of the display device that would normally trigger a notification being generated and output to the patient.
Embodiment 3: The method of Embodiments 1-2, further comprising: upon determining that the patient is not currently experiencing the analyte state corresponding to the alert state of the display device that would normally trigger the notification being generated and output to the patient, resetting, by the display device, at least one of: an alert timer initiated by the display device prior to the signal loss event, the alert state of the display device set prior to the signal loss event, or an alert state mode of the display device set prior to the signal loss event.
Embodiment 4: The method of Embodiments 1-3, wherein resetting the alert timer comprises starting a new alert timer for a full or partial time period corresponding with the new timer.
Embodiment 5: The method of Embodiments 1-4, wherein resetting the alert state comprises setting the alert state to a normal alert state corresponding to a normal nominal analyte concentration value range of a patient.
Embodiment 6: The method of Embodiments 1-5, wherein resetting the alert state mode comprises setting the alert state mode to an idle mode where no alert is generated and output to a patient.
Embodiment 7: The method of Embodiments 1-6, further comprising: upon determining that the patient is currently experiencing the analyte state corresponding to the alert state of the display device that would normally trigger the notification being generated and output to the patient, determining, by the display device, whether the signal loss event has a duration less than a threshold maximum amount of time.
Embodiment 8: The method of Embodiments 1-7, further comprising: upon determining that the signal loss event does not have a duration less than the threshold maximum amount of time, resetting, by the display device, at least one of: an alert timer initiated by the display device prior to the signal loss event, the alert state of the display device set prior to the signal loss event, or an alert state mode of the device set prior to the signal loss event.
Embodiment 9: The method of Embodiments 1-8, wherein resetting the alert timer comprises starting a new alert timer for a full or partial time period corresponding with the new timer.
Embodiment 10: The method of Embodiments 1-9, wherein resetting the alert state comprises setting the alert state to a normal alert state corresponding to a normal nominal analyte concentration value range of a patient.
Embodiment 11: The method of Embodiments 1-10, wherein resetting the alert state mode comprises setting the alert state mode to an idle mode where no alert is generated and output to a patient.
Embodiment 12: The method of Embodiments 1-11, wherein resetting the at least one of the alert timer, the alert state, or the alert state mode is based on the current analyte data and not the backfill data.
Embodiment 13: The method of Embodiments 1-12, further comprising: upon determining that the signal loss event does have a duration less than the threshold maximum amount of time, determining, by the display device, at least one of: whether the display device was operating in an acknowledged alert mode upon onset of the signal loss event, or whether the alert state of the display device that would normally trigger the notification being generated and output to the patient is associated with a timer.
Embodiment 14: The method of Embodiments 1-13, further comprising: upon determining that the display device was operating in an acknowledged alert mode upon onset of the signal loss event or that the alert state of the display device that would normally trigger the notification being generated and output to the patient is associated with the timer, setting, by the display device, at least one of: a current alert timer, the current alert state of the display device, or a current alert state mode of the display device.
Embodiment 15: The method of Embodiments 1-14, wherein setting the at least one of the current alert timer, the current alert state, or the current alert state mode is based on the current analyte data and the backfill data.
Embodiment 16: The method of Embodiments 1-15, wherein processing at least the current analyte data to determine the current alert state of the display device comprises processing the current analyte data and the backfill data, or the current analyte data and not the backfill data.
Embodiment 17: An analyte monitoring system comprising: a sensor system, comprising: a continuous analyte sensor configured to measure an analyte concentration of a patient; a sensor electronics module configured to: receive a signal from the continuous analyte sensor that is indicative of the analyte concentration; generate analyte data based on the signal; and transmit, via a wireless transceiver, the analyte data to at least a display device; and the display device in direct wireless communication with the sensor electronics module, comprising: one or more memories; and one or more processors communicatively coupled to the one or more memories, the one or more processors configured to: receive, upon recovery of wireless communication between the display device and the sensor electronics module after a signal loss event, historical analyte data generated by the sensor electronics module during the signal loss event; receive, from the sensor electronics module, current analyte data generated by the sensor electronics module after the signal loss event; and determine a current alert state of the display device based on at least one of the received historical analyte data or the current analyte data.
Embodiment 18: The analyte monitoring system of Embodiment 17, wherein determining the current alert state of the display device comprises processing the current analyte data and the backfill data, or processing the current analyte data and not the backfill data.
Embodiment 19: The analyte monitoring system of Embodiments 17-18, wherein determining the current alert state of the display device comprises: determining whether the patient is currently experiencing an analyte state corresponding to an alert state of the display device that would normally trigger a notification to be generated and output to the patient.
Embodiment 20: The analyte monitoring system of Embodiments 17-19, wherein determining the current alert state of the display device further comprises: upon determining that the patient is not currently experiencing the analyte state corresponding to the alert state of the display device that would normally trigger the notification being generated and output to the patient, resetting, by the display device, at least one of: an alert timer initiated by the display device prior to the signal loss event, the alert state of the display device set prior to the signal loss event, or an alert state mode of the display device set prior to the signal loss event.
Embodiment 21: The analyte monitoring system of Embodiments 17-20, wherein determining the current alert state of the display device further comprises: upon determining that the patient is currently experiencing the analyte state corresponding to the alert state of the display device that would normally trigger the notification being generated and output to the patient, determining, by the display device, whether the signal loss event has a duration less than a threshold maximum amount of time.
Embodiment 22: The analyte monitoring system of Embodiments 17-21, wherein determining the current alert state of the display device further comprises: upon determining that the signal loss event does not have a duration less than the threshold maximum amount of time, resetting, by the display device, at least one of: an alert timer initiated by the display device prior to the signal loss event, the alert state of the display device set prior to the signal loss event, or an alert state mode of the device set prior to the signal loss event.
Embodiment 23: The analyte monitoring system of Embodiments 17-22, further comprising: upon determining that the signal loss event does have a duration less than the threshold maximum amount of time, determining, by the display device, at least one of: whether the display device was operating in an acknowledged alert mode upon onset of the signal loss event, or whether the alert state of the display device that would normally trigger the notification being generated and output to the patient is associated with a timer.
Embodiment 24: The analyte monitoring system of Embodiments 17-23, further comprising: upon determining that the display device was operating in an acknowledged alert mode upon onset of the signal loss event or that the alert state of the display device that would normally trigger the notification being generated and output to the patient is associated with the timer, setting, by the display device, at least one of: a current alert timer, the current alert state of the display device, or a current alert state mode of the display device.
Embodiment 25: A method for providing improved data accuracy associated with a display device of an analyte monitoring system, comprising: re-establishing a wireless communication signal between a first display device and an analyte sensor system after a signal loss event; upon re-establishing the wireless communication signal, receiving, by the first display device, backfill data from at least one of the analyte sensor system or a second display device, the backfill data comprising historical analyte data of the patient collected by the analyte sensor system during the signal loss event; and processing, by the first display device, the backfill data to determine a current alert state of the first display device.
Embodiment 26: The method of Embodiment 25, wherein the backfill data is received by the first display device from the second display device.
Embodiment 27: The method of Embodiments 25-26, wherein the first display device comprises a smart phone and the second display device comprises a smart watch.
Embodiment 28: The method of Embodiments 25-27, further comprising: upon re-establishing the wireless communication signal, receiving, by the first display device, current analyte data of a patient from at least one of the analyte sensor system or the second display device; and processing, by the first display device, the current analyte data with the backfill data to determine the current alert state of the first display device.
Embodiment 29: The method of Embodiments 25-28, wherein at least one of the backfill data or the current analyte data is received by the first display device from the second display device.
Embodiment 30: The method of Embodiments 25-29, wherein the first display device comprises a smart phone and the second display device comprises a smart watch.
Embodiment 31: The method of Embodiments 25-30, further comprising: determining that the signal loss event meets a minimum time threshold prior to the first display device receiving or processing the backfill data.
Embodiment 32: The method of Embodiments 25-31, wherein determining the current alert state of the first display device comprises at least one of: starting a new alert timer for a full or partial time period corresponding with the new timer; continuing an alert timer initiated prior to the signal loss event; cancelling the alert timer initiated prior to the signal loss event; changing an alert state set prior to the signal loss event; or continuing operation in the alert state set prior the signal loss event.
Embodiment 33: The method of Embodiments 25-32, wherein processing the backfill data comprises evaluating all of the historical analyte data collected by the at least one of the analyte sensor system or the second display device during the signal loss event.
Embodiment 34: The method of Embodiments 25-33, wherein processing the backfill data comprises evaluating only a portion of the historical analyte data collected by the at least one of the analyte sensor system or the second display device during the signal loss event.
Embodiment 35: An analyte monitoring system comprising: a sensor system, comprising: a continuous analyte sensor configured to measure an analyte concentration of a patient; a sensor electronics module configured to: receive a signal from the continuous analyte sensor that is indicative of the analyte concentration; generate analyte data based on the signal; and transmit, via a wireless transceiver, the analyte data to a first display device and a second display device; the first display device in direct wireless communication with the sensor electronics module, comprising: one or more memories; and one or more processors communicatively coupled to the one or more memories, the one or more processors configured to: receive, upon recovery of wireless communication between the first display device and the sensor electronics module after a signal loss event, historical analyte data generated by the sensor electronics module during the signal loss event from at least one of the sensor electronics module or the second display device; receive, from at least one of the sensor electronics module or the second display device, current analyte data generated by the sensor electronics module after the signal loss event; and determine a current alert state of the first display device based on at least one of the received historical analyte data or the current analyte data; and the second display device in direct wireless communication with the sensor electronics module and the first display device.
Embodiment 36: The analyte monitoring system of Embodiment 35, wherein determining the current alert state of the first display device is based on both of the received historical analyte data and the current analyte data.
Embodiment 37: The analyte monitoring system of Embodiments 35-36, wherein the one or more processors are configured to: automatically process and analyze all of the historical data analyte data generated by the sensor electronics module during the signal loss event upon receiving the historical analyte data.
Embodiment 38: The analyte monitoring system of Embodiments 35-37, wherein determining the current alert state of the first display device is based on the current analyte data and not the received historical analyte data.
Embodiment 39: The analyte monitoring system of Embodiments 35-38, wherein the one or more processors are further configured to: determine whether a notification triggering event occurred after the signal loss event, wherein: responsive to determining that the notification triggering event did occur after the signal loss event, the one or more processors are configured to determine the current alert state of the first display device based on both of the received historical analyte data and the current analyte data; and responsive to determining that the notification triggering event did not occur after the signal loss event, the one or more processors are configured to determine the current alert state of the first display device based on the received current analyte data and not the historical analyte data.
Embodiment 40: The analyte monitoring system of Embodiments 35-39, wherein the one or more processors are further configured to: determine an onset of the signal loss event based on a deviation from an expected frequency of receiving the analyte data from the sensor electronics module.
Embodiment 41: The analyte monitoring system of Embodiments 35-40, wherein the sensor electronics module is further configured to: store the generated analyte data; and in response to receiving a request for the historical analyte data by the one or more processors, transmit at least a portion of the historical analyte data to the first display device.
Embodiment 42: The analyte monitoring system of Embodiments 35-41, wherein the second display device is further configured to: receive and store the generated analyte data; and in response to receiving a request for the historical analyte data by the one or more processors, transmit at least a portion of the historical analyte data to the first display device.
Embodiment 43: The analyte monitoring system of Embodiments 35-42, wherein each of the sensor electronics module and the second display device is further configured to: receive and store at least a portion of the generated analyte data; and in response to receiving a request for the historical analyte data by the one or more processors, transmit the at least the portion of the historical analyte data to the first display device.
Embodiment 44: The analyte monitoring system of Embodiments 35-43, wherein: the first display device comprises a smart phone; and the second display device comprises a smart watch.

What is claimed is:

1. A method for ensuring an accuracy of a display device of an analyte monitoring system, comprising:
re-establishing a wireless communication signal between a display device and an analyte sensor system after a signal loss event;
upon re-establishing the wireless communication signal, receiving, by the display device, backfill data from the analyte sensor system, the backfill data comprising historical analyte data of the patient collected by the analyte sensor system during the signal loss event; and
processing, by the display device, the backfill data to determine a current alert state of the display device.

2. The method of claim 1, further comprising:
upon re-establishing the wireless communication signal, receiving, by the display device, current analyte data of a patient from the analyte sensor system; and
processing, by the display device, the current analyte data with the backfill data to determine the current alert state of the display device.

3. The method of claim 1, further comprising:
determining that the signal loss event meets a minimum time threshold prior to the display device receiving or processing the backfill data from the analyte sensor system.

4. The method of claim 1, wherein determining the current alert state of the display device comprises at least one of:
starting a new alert timer for a full or partial time period corresponding with the new timer;
continuing an alert timer initiated prior to the signal loss event;
cancelling the alert timer initiated prior to the signal loss event;
changing an alert state set prior to the signal loss event; or
continuing operation in the alert state set prior the signal loss event.

5. The method of claim 1, wherein processing the backfill data comprises evaluating all of the historical analyte data collected by the analyte sensor system during the signal loss event.

6. The method of claim 1, wherein processing the backfill data comprises evaluating only a portion of the historical analyte data collected by the analyte sensor system during the signal loss event.

7. The method of claim 1, wherein processing the backfill data comprises evaluating the backfill data in forward chronological order.

8. The method of claim 1, wherein processing the backfill data comprises evaluating the backfill data in reverse chronological order.

9. An analyte monitoring system comprising:
a sensor system, comprising:
a continuous analyte sensor configured to measure an analyte concentration of a patient;
a sensor electronics module configured to:
receive a signal from the continuous analyte sensor that is indicative of the analyte concentration;
generate analyte data based on the signal; and
transmit, via a wireless transceiver, the analyte data to at least a first display device; and
the first display device in direct wireless communication with the sensor electronics module, comprising:
one or more memories; and
one or more processors communicatively coupled to the one or more memories, the one or more processors configured to ensure an accuracy of the first display device by determining a current alert state of the first display device based on at least one of received historical analyte data or current analyte data,
wherein:
upon recovery of wireless communication between the first display device and the sensor electronics module after a signal loss event, the historical analyte data generated by the sensor electronics module during the signal loss event is received from the sensor electronics module by the one or more processors, and
current analyte data generated by the sensor electronics module after the signal loss event is received from the sensor electronics module by the one or more processors.

10. The analyte monitoring system of claim 9, wherein determining the current alert state of the first display device is based on both of the received historical analyte data and the current analyte data.

11. The analyte monitoring system of claim 10, wherein the one or more processors are configured to:
automatically process and analyze all of the historical data analyte data generated by the sensor electronics module during the signal loss event upon receiving the historical analyte data.

12. The analyte monitoring system of claim 9, wherein the one or more processors are further configured to:
determine whether a notification triggering event occurred after the signal loss event, wherein:
responsive to determining that the notification triggering event did occur after the signal loss event, the one or more processors are configured to determine the current alert state of the first display device based on both of the received historical analyte data and the current analyte data; and
responsive to determining that the notification triggering event did not occur after the signal loss event, the one or more processors are configured to determine the current alert state of the first display device based on the received current analyte data and not the historical analyte data.

13. The analyte monitoring system of claim 9, wherein the one or more processors are further configured to:
determine an onset of the signal loss event based on a deviation from an expected frequency of receiving the analyte data from the sensor electronics module.

14. The analyte monitoring system of claim 9, wherein the sensor electronics module is further configured to:
store the generated analyte data; and
in response to receiving a request for the historical analyte data by the one or more processors, transmit at least a portion of the historical analyte data to the first display device.

15. The analyte monitoring system of claim 14, further comprising:
a second display device in direct wireless communication with the sensor electronics module, wherein the sensor electronics module is further configured to transmit, using the wireless transceiver, the analyte data directly to the second display device.

16. The analyte monitoring system of claim 15, wherein:
during the signal loss event, the second display device is configured to receive and store at least a portion of the historical analyte data generated by the sensor electronics module during the signal loss event.

17. The analyte monitoring system of claim 16, wherein:
in response to receiving a request for the historical analyte data by the one or more processors, the second display device is configured to transmit the at least a portion of the historical analyte data to the first display device.

18. The analyte monitoring system of claim 17, wherein:
the first display device comprises a smart phone; and
the second display device comprises a smart watch.

19. The analyte monitoring system of claim 9, wherein determining the current alert state of the first display device comprises processing the historical analyte data in chronological order.

20. The analyte monitoring system of claim 9, wherein determining the current alert state of the first display device comprises processing the historical analyte data in reverse chronological order.
